(19) United States
(12) Patent Application Publication
(10) Pub. No.: US 2025/0089832 A1
(43) Pub. Date: Mar. 20, 2025

(54) HAIR BONNET

(71) Applicant: Nothing But Net International, LLC, Wilmington, DE (US)

(72) Inventors: Treveen Neikita Stewart, Encino, CA (US); Tamara Hill, West Bay (KY)

(73) Assignee: Nothing But Net International, LLC, Wilmington, DE (US)

(21) Appl. No.: 18/799,199

(22) Filed: Aug. 9, 2024

Related U.S. Application Data
(60) Provisional application No. 63/538,771, filed on Sep. 15, 2023.

(51) Int. Cl. A42B 1/049; A61F 9/04

(52) U.S. Cl. CPC A42B 1/049; A61F 9/04

(57) ABSTRACT

A hair bonnet includes a protective hair covering comprised of fabric. The hair bonnet also includes an eye mask coupled to the protective hair covering.

### CROSS-REFERENCE TO RELATED APPLICATIONS

This application claims benefit under 35 U.S.C. § 119 (e) of the U.S. Provisional Application No. 63/538,771 filed Sep. 15, 2023, the content of which is incorporated herein by reference in its entirety.

### TECHNICAL FIELD

This disclosure relates generally to head coverings, and more specifically, to hair bonnets.

### BACKGROUND

Hair bonnets are often worn by users while sleeping to retain moisture and natural oils in a user's hair and minimize friction between the user's hair and other surfaces, such as the user's pillow. However, traditional hair bonnets are not adjustable, do not include an eye mask, and do not utilize breathable fabrics. As such, traditional hair bonnets are often uncomfortable to wear and often slip off of the user's head while sleeping, causing their hair to tangle and lose moisture at night. Therefore, a solution is needed to address these shortcomings.

### SUMMARY

Embodiments of the invention solve these problems by providing a hair bonnet that is comfortable, adjustable, and able to stay on a user's head throughout the night.

In one embodiment of the invention, a hair bonnet includes a protective hair covering comprised of fabric. The hair bonnet also includes an eye mask coupled to the protective hair covering.

In another embodiment of the invention, a kit for a hair bonnet includes a hair bonnet. The hair bonnet includes a protective hair covering comprised of fabric. The hair bonnet also includes an eye mask coupled to the protective hair covering. The eye mask includes an opening.

### BRIEF DESCRIPTION OF THE DRAWINGS

Disclosed herein are embodiments of systems and apparatuses pertaining to hair bonnets. This description includes drawings, wherein:

FIG. 1 is a side view of a hair bonnet, according to some embodiments.

FIG. 2 is a front view of the hair bonnet of FIG. 1.

FIG. 3 is a perspective view of the hair bonnet of FIG. 1.

FIG. 4 is an interior view of a hair bonnet, according to some embodiments.

FIGS. 5A-5B are perspective views of the hair bonnet of FIG. 4.

FIG. 6 is a perspective, detailed view of a hair bonnet, according to some embodiments.

FIG. 7 is a block diagram of a kit for a hair bonnet, according to some embodiments.

FIG. 8 is a front view of a hair bonnet being worn by a user, according to another embodiment.

FIG. 9 is a side view of the hair bonnet of FIG. 8 worn by a user.

FIG. 10 is a rear view of the hair bonnet of FIG. 8 worn by a user.

FIG. 11 is a perspective view of the hair bonnet of FIG. 8.

FIG. 12 is a perspective view of the hair bonnet of FIG. 8 with an insert partially removed.

FIG. 13 is a front view of the hair bonnet of FIG. 8.

FIG. 14 is a rear view of the hair bonnet of FIG. 8.

FIG. 15 is a front view of the hair bonnet of FIG. 8 with an insert partially removed.

FIG. 16 is a perspective view of a hair bonnet being worn by a user, according to another embodiment.

FIG. 17 is a right side view of the hair bonnet of FIG. 16.

FIG. 18 is a left side view of the hair bonnet of FIG. 16.

FIG. 19 is a front view of the hair bonnet of FIG. 16.

FIG. 20 is a front view of the hair bonnet of FIG. 16 with tied straps.

FIG. 21 is a rear view of a hair bonnet, according to yet another embodiment.

FIG. 22 is a perspective view of the hair bonnet of FIG. 21.

FIG. 23 is a perspective view of a hair bonnet worn by a user, according to one embodiment.

FIG. 24 is a left side view of the hair bonnet of FIG. 23, worn by a user.

FIG. 25 is a right side view of the hair bonnet of FIG. 23, worn by a user.

FIG. 26 is a front view of the hair bonnet of FIG. 23, worn by a user.

FIG. 27 is a rear view of the hair bonnet of FIG. 23, worn by a user.

FIG. 28 is a front view of the hair bonnet of FIG. 23.

FIG. 29 is a perspective view of the hair bonnet of FIG. 23.

FIG. 30 is a perspective view of a hair bonnet, according to another embodiment.

FIG. 31 is a right side view of the hair bonnet of FIG. 30, worn by a user.

FIG. 32 is a left side view of the hair bonnet of FIG. 30, worn by a user.

FIG. 33 is a front view of the hair bonnet of FIG. 30, worn by a user.

FIG. 34 is a rear view of the hair bonnet of FIG. 30, worn by a user.

FIG. 35 is a side view of the hair bonnet of FIG. 30, worn by a user.

FIG. 36 is a perspective view of the hair bonnet of FIG. 30.

FIG. 37 is a front view of a hair bonnet worn by a user, according to yet another embodiment.

FIG. 38 is a right side view of the hair bonnet of FIG. 37, worn by a user.

FIG. 39 is a left side view of the hair bonnet of FIG. 37, worn by a user.

FIG. 40 is a rear view of the hair bonnet of FIG. 37, worn by a user.

FIG. 41 is a perspective view of the hair bonnet of FIG. 37, with untied straps.

FIG. 42 is a perspective view of the hair bonnet of FIG. 37, with tied straps.

FIG. 43 is a rear view of the hair bonnet of FIG. 37.

FIG. 44 is a front view of a hair bonnet worn by a user, according to another embodiment.

FIG. 45 is a right side view of the hair bonnet of FIG. 44, worn by a user.

FIG. 46 is a rear view of the hair bonnet of FIG. 44, worn by a user.

FIG. 47 is a right side view of the hair bonnet of FIG. 44.

FIG. 48 is a left side view of the hair bonnet of FIG. 44.

FIG. 49 is a perspective view of the hair bonnet of FIG. 44.

FIG. 50 is a rear view of the hair bonnet of FIG. 44.

FIG. 51 is a front view of a hair bonnet worn by a user, according to yet another embodiment.

FIG. 52 is a rear view of the hair bonnet of FIG. 51, worn by a user.

FIG. 53 is a right side view of the hair bonnet of FIG. 51, worn by a user.

FIG. 54 is a left side view of the hair bonnet of FIG. 51, worn by a user.

FIG. 55 is a front view of the hair bonnet of FIG. 51.

FIG. 56 is a rear view of the hair bonnet of FIG. 51.

FIG. 57 is a bottom view of the hair bonnet of FIG. 51.

FIG. 58 is a perspective view of a hair bonnet worn by a user, according to another embodiment.

FIG. 59 is a right side view of the hair bonnet of FIG. 58, worn by a user.

FIG. 60 is a front view of the hair bonnet of FIG. 58, worn by a user.

FIG. 61 is a right side view of the hair bonnet of FIG. 58, worn by a user and with an eye mask flipped upwards.

FIG. 62 is a left side view of the hair bonnet of FIG. 58, worn by a user and with an eye mask flipped upwards.

FIG. 63 is a right side view of the hair bonnet of FIG. 58, worn by a user and with an eye mask flipped upwards.

FIG. 64 is a right side view of the hair bonnet of FIG. 58.

### DETAILED DESCRIPTION

Embodiments of the invention are described with reference to the attached figures, wherein like reference numerals are used throughout the figures to designate similar or equivalent elements. The figures are not drawn to scale and they are provided merely to illustrate the instant invention. Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details or with other methods. In other instances, well-known structures or operations are not shown in detail to avoid obscuring the invention. The present invention is not limited by the illustrated ordering of acts or events, as some acts may occur in different orders and/or concurrently with other acts or events. Furthermore, not all illustrated acts or events are required to implement a methodology in accordance with the present invention.

Embodiments of the invention provide a unique and innovative wellness eye mask bonnet designed to provide users with the much-needed hair health, strand protection, enhanced rest, and relaxation experience. The bonnet is configured to be a hair shield, protecting hairstyles with moisture retention, maintaining the natural levels of the hair's natural oil, allowing less friction between the user's hair and other surfaces, minimizing hair manipulation whilst tossing and turning, promoting hair health, all the while being utilized as a mask that allows users to sooth eyes, fall asleep fast and reach deep REM to provide a good night's rest and a better day ahead. The eye mask bonnet aims to address the limitations of the prior art by offering a complete coverage solution for hair and eye, promoting a much-needed peaceful rest and rejuvenation for the hair and eye of the wearer. Embodiments of the invention may also be used for home relaxation, travel, sleep disorders management, meditation practices, wellness centers or spas and other environments to provide restful, quiet, and/or serene experiences.

As used herein, the term “hair bonnet” refers to any cap, hat, durag, beanie, bonnet, hood, scarf, or other head covering that assists in retaining moisture in hair, compresses hair, minimizes tangles in hair, and/or minimizes hair damage.

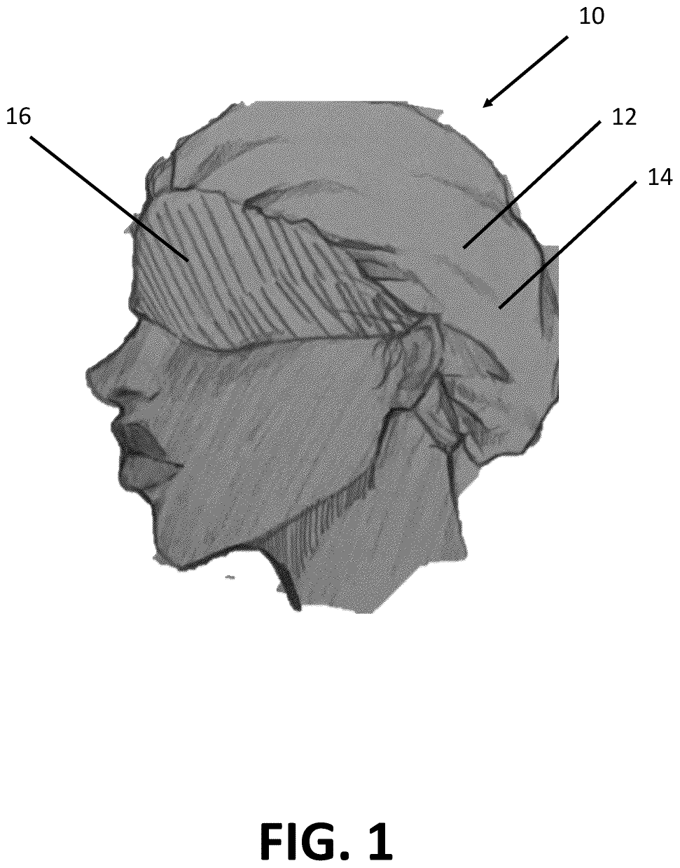

FIG. 1 is a side view of a hair bonnet 10, according to some embodiments. The hair bonnet 10 includes a protective hair covering 12 and an eye mask 16 coupled to the protective hair covering 12.

The protective hair covering 12 is made, at least in part, of fabric 14. In embodiments, the protective hair covering 12 is crafted from recyclable, soft, hypoallergenic, noncomedogenic and breathable materials to ensure clean and maximum comfort during use. The chosen materials are desirably gentle on the skin, preventing irritation or discomfort, even during prolonged wear. Examples of suitable fabrics 14 include, but are not limited to, satin, polyester, acrylic silk, nylon, and/or bamboo fabric. In some embodiments, the protective hair covering is configured to prevent moisture loss and/or prevent hair breakage. For example, and in some embodiments, the protective hair covering 12 is hydrophobic to assist in preventing moisture loss and/or hair breakage.

In some embodiments, the protective hair covering 12 is configured to completely cover a user's hair. For example, the protective hair covering 12 may be shaped and sized so that the user's hair is completely enveloped.

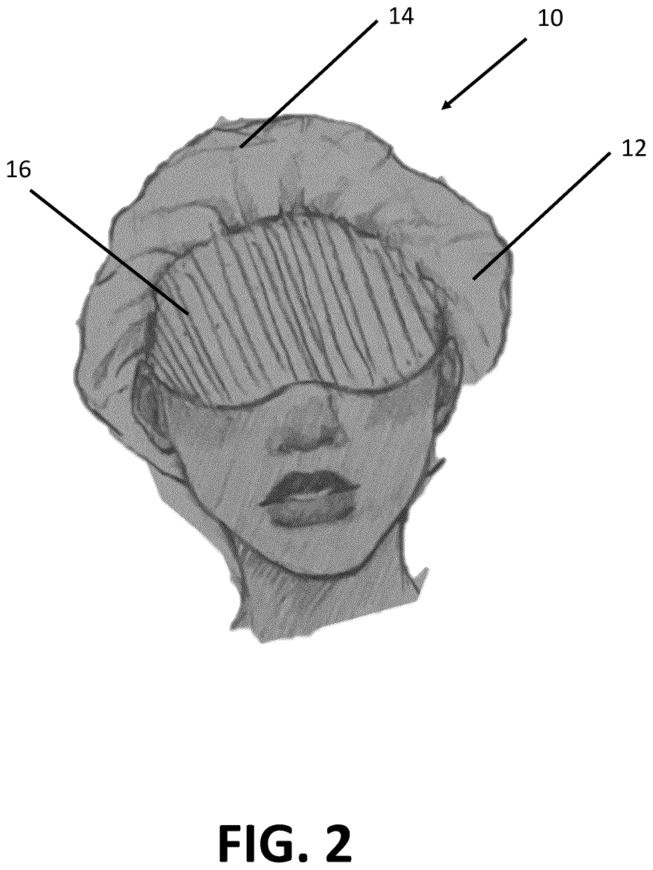

FIG. 2 is a front view of the hair bonnet 10 of FIG. 1. According to some embodiments, and as shown in FIG. 2, the eye mask 16 is integral with the protective hair covering 12. The eye mask 16 being integral with the protective hair covering 12 may be accomplished, for example, by the eye mask 16 and the protective hair covering 12 being sewn together or by the eye mask 16 and the protective hair covering 12 being monolithic. However, this is not required, and in some embodiments, the eye mask 16 is removably coupled to the protective hair covering 12. For example, and in some embodiments, the eye mask 16 and/or the protective hair covering 12 may include snaps, buttons and holes, hook and loop fasteners, G-hooks and loops, S-hooks and loops, or any other suitable fasteners, to removably couple the eye mask 16 to the protective hair covering 12.

The eye mask 16 is equipped with light-blocking technology. This feature ensures that external light does not interfere with the wearer's tranquility, facilitating a restful and undisturbed experience. It is a sleep mask that encourages deep REM sleep anywhere—at home, outdoors and travel. In some embodiments, the eye mask 16 is configured to block at least 50% of light, at least 60% of light, at least 70% of light, at least 80% of light, and/or at least 90% of light. For example, the eye mask 16 blocking light may assist a wearer of the hair bonnet 10 in falling and/or staying asleep.

In some embodiments, the eye mask 16 is made, at least in part, of fabric. In some embodiments, the eye mask 16 is crafted from recyclable, soft, hypoallergenic, noncomedogenic and breathable materials to ensure clean and maximum comfort during use. The chosen materials are desirably gentle on the skin, preventing irritation or discomfort, even during prolonged wear. Examples of suitable fabrics for the eye mask 16 include, but are not limited to, satin, polyester, acrylic silk, nylon, and/or bamboo fabric. In some embodiments, the fabric of the eye mask is the same as the fabric of the hair protective hair covering 12. In some embodiments, the eye mask 16 includes padding. For example, padding may be useful for making the eye mask 16 more comfortable for a user.

As can be seen in FIGS. 1 and 2, the eye mask 16 is relatively tight compared to the protective hair covering 12 to snugly and securely fit on the user's head. In some embodiments, the eye mask 16 and/or edge of the protective hair covering 12 includes elastic to securely fit the hair bonnet 10 on the user's head. The eye mask 16 being relatively tight assists the hair bonnet 10 in remaining on a user's head throughout an entire night's sleep. By remaining on a user's head throughout the night, the hair bonnet 10 is able to minimize hair tangling and minimize moisture loss from the user's hair. The protective hair covering 12 is relatively loose to avoid matting the hair of the user and avoid creating friction with the user's hair. The combination of the protective hair covering 12 and eye mask 16 provides a cocoon-like environment for deep relaxation. This comprehensive coverage helps block out any sources of disturbance and creates a serene atmosphere conducive to happy hair, restful sleep, and pure relaxation. The lightweight, form-fitting design that covers the eyes (without pressing on them) provides a fit that is comfortable on the user's face and head, but also ensures the hair bonnet 10 is secured on a user's face and head.

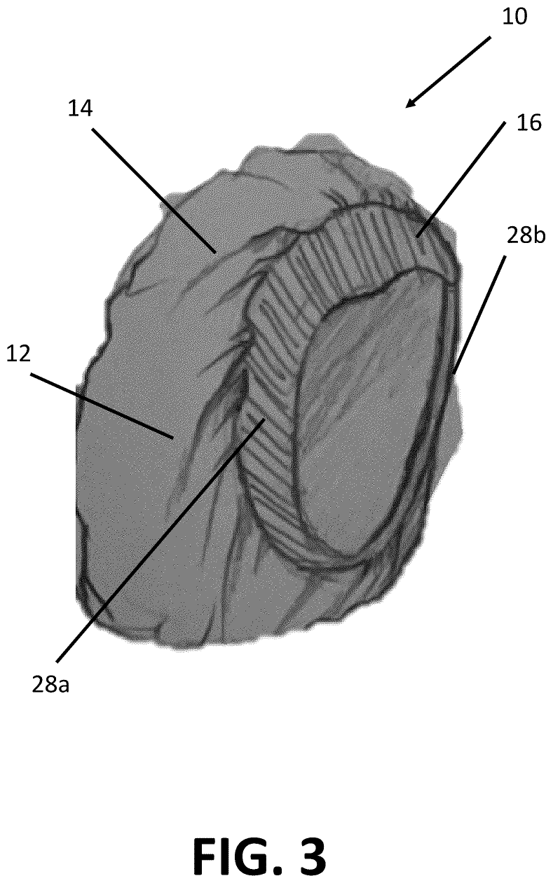

FIG. 3 is a perspective view of the hair bonnet 10 of FIG. 1. According to some embodiments, and as shown, for example, in FIG. 3, the hair bonnet 10 may be adapted to include acoustic dampening elements that function as an ear mask 28a, 28b. For example, the hair bonnet 10 may include an opening designed to removably receive an acoustic dampening element at a location corresponding to the user's ear to form the ear mask 28a, 28b. In an alternative embodiment, the ear mask 28a, 28b may be a separate feature coupled to one or more of the protective hair covering 12 and the eye mask 16. In some embodiments, one or more ear masks 28a, 28b are spaced and sized to cover one or more ears of a user. In some embodiments, the ear mask 28a, 28b is integral with the protective hair covering 12. In some embodiments, the ear mask 28a, 28b is removably coupled to the protective hair covering 12. For example, and in some embodiments, the ear mask 28a, 28b and/or the protective hair covering 12 may include snaps, buttons and holes, hook and loop fasteners, G-hooks and loops, S-hooks and loops, or any other suitable fasteners, to removably couple the ear mask 28a, 28b to the protective hair covering 12.

In some embodiments, the ear masks 28a, 28b are made of fabric. Examples of suitable fabrics include, but are not limited to, satin, polyester, acrylic silk, nylon, and/or bamboo fabric. In some embodiments, the ear mask 28a, 28b is configured to block at least 50%, at least 60%, at least 70%, at least 80%, and/or at least 90% of sound. Blocking sound may be useful for facilitating a user of the hair bonnet 10 in falling and/or staying asleep.

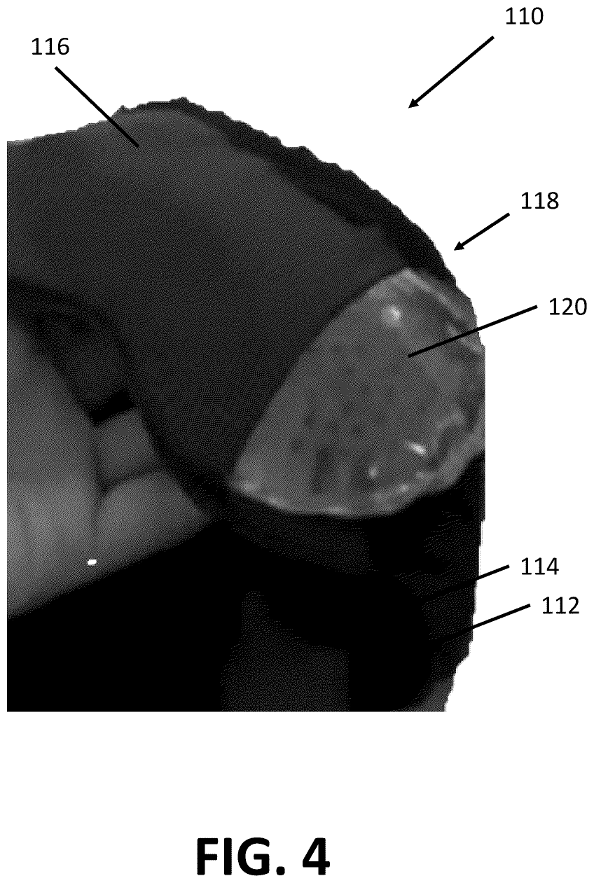

FIG. 4 is an interior view of a hair bonnet 110, according to some embodiments. The hair bonnet 110 includes a protective hair covering 112 made, at least in part, of fabric 114. An eye mask 116 is coupled to the protective hair covering 112. In some embodiments, the eye mask 116 includes and defines an opening 118. An insert 120 may be removably inserted into the opening 118. In some embodiments, the insert 120 includes a gel eye mask, a cooling pack, and/or a heating pack. In some embodiments, the insert 120 includes additional padding to increase the comfortability of the hair bonnet 110. The removable insert 120 provides additional relaxation benefits, such as soothing tired eyes, puffy eyes, and/or dark circles, and can also be used as a warm compress to relieve eye fatigue and stimulate circulation. The removable insert 120 also promotes better sleep through temperature regulation.

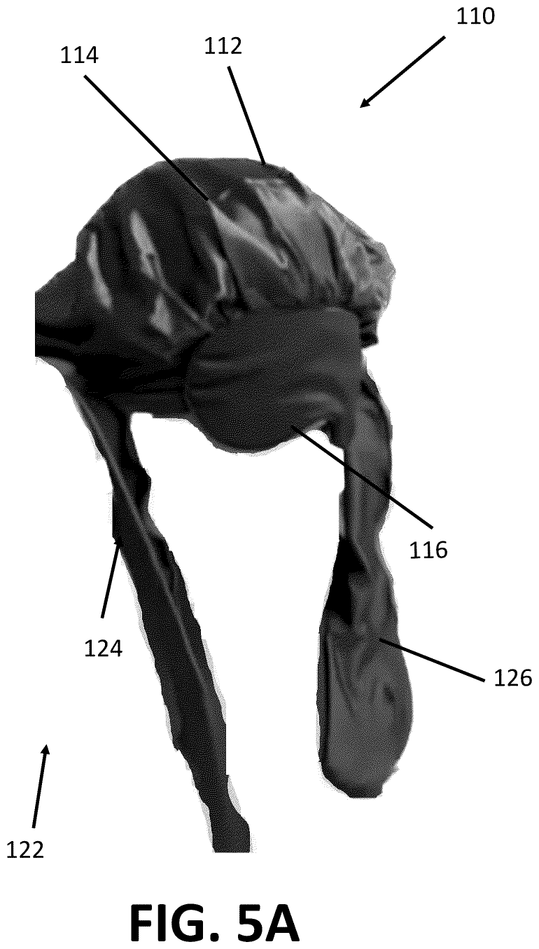

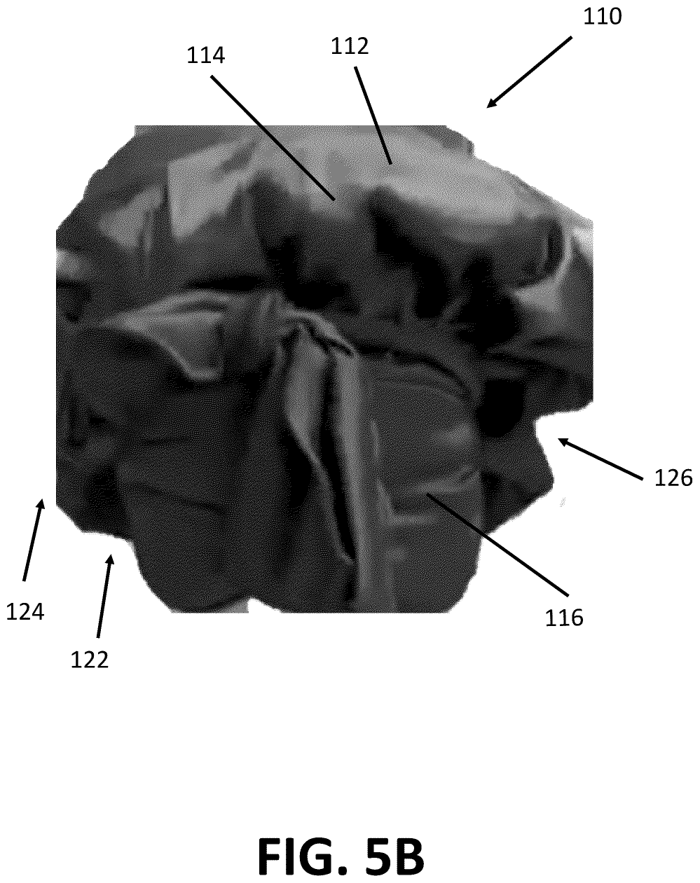

FIGS. 5A-5B are perspective views of the hair bonnet 110 of FIG. 4. In some embodiments, and as shown, for example, in FIG. 5, the hair bonnet 110 includes an adjustable securing mechanism 122 coupled to the protective hair covering 112. The adjustable securing mechanism 122 may be useful for securing the hair bonnet 110 to the head of a user. For example, the securing mechanism 112 may allow users to achieve a personalized, snug fit. The securing mechanism 112 ensures that the bonnet 110 remains in place throughout the duration of use, preventing any unintended movements that could disrupt the wearer's relaxation through extended wear.

In some embodiments, as shown in FIG. 5A, the securing mechanism 122 includes a first strap 124 and a second strap 126. In some embodiments, the first strap 124 and/or the second strap 126 comprise a same fabric 114 as that of the protective hair covering 112. In some embodiments, the first strap 124 and/or the second strap 126 are made of fabric. Examples of suitable fabrics include, but are not limited to, satin, polyester, acrylic, silk, satin, and/or bamboo fabric. In some embodiments, the first strap 124 and/or the second strap 126 additionally include elastic. In some embodiments, the first strap 124 and the second strap 126 are made of the same material. However, this is not required and in other embodiments, the first strap 124 and the second strap 126 are made of different material.

In some embodiments, the first strap 124 and the second strap 126 are configured to wrap around a head of a user and tie together to securely hold the hair bonnet 110 on a user's head, as shown in FIG. 5B. For example, and in some embodiments, the first strap 124 and the second strap 126 extend from the hair bonnet 110 at the nape of the user's neck. To secure the hair bonnet 110, the first strap 124 and the second strap 126 may be pulled back around the user's neck, wrap back around towards the forehead of the user and be tied together (e.g., in a bow) above the eye mask 116. In another embodiment, the first strap 124 and the second strap 126 may wrap around the forehead of the user and be tied together (e.g., in a bow) at the nape of the user's neck.

In some embodiments, the first strap 124 and/or the second strap 126 are at least 12 inches, at least 14 inches, at least 16 inches, at least 18 inches, at least 20 inches, at least 22 inches, and/or at least 24 inches in length.

In some embodiments, the first strap 124 and the second strap 126 may be removably secured to each other via snaps, buttons and holes, hook and loop fasteners, G-hooks and loops, S-hooks and loops, or any other suitable fasteners.

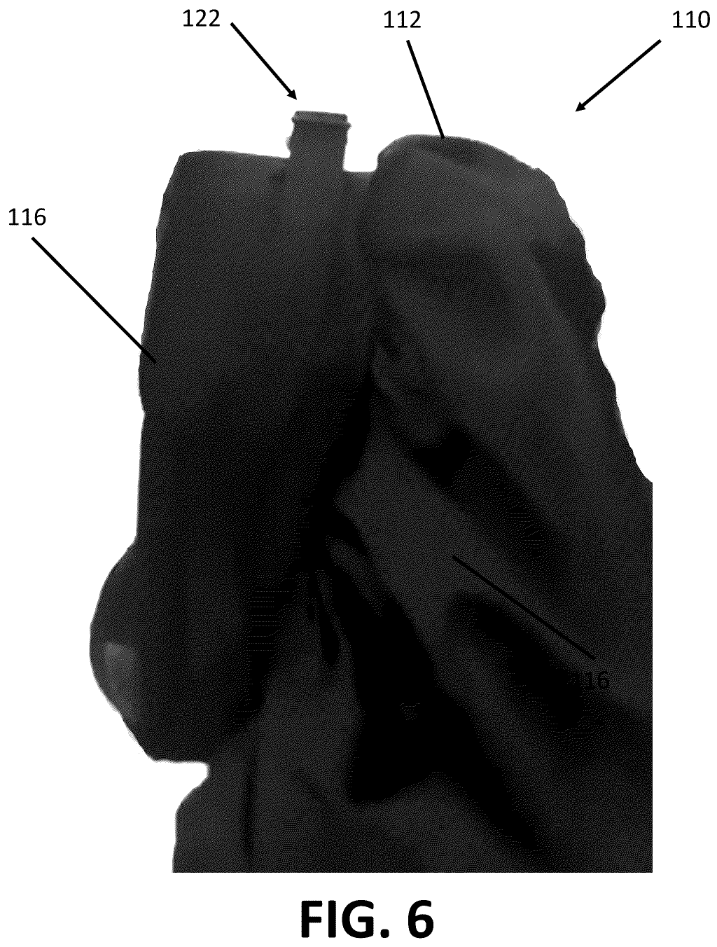

FIG. 6 illustrates an alternative securing mechanism 122 for securing the hair bonnet 110 to the head of the user, according to some embodiments. As shown in FIG. 6, the eye mask 116 may include an adjustable strap/clasp mechanism. A user can slide the elastic grip to change the amount of tension on the eye mask to secure both the eye mask 116 and protective hair covering 112 in place on the user's head.

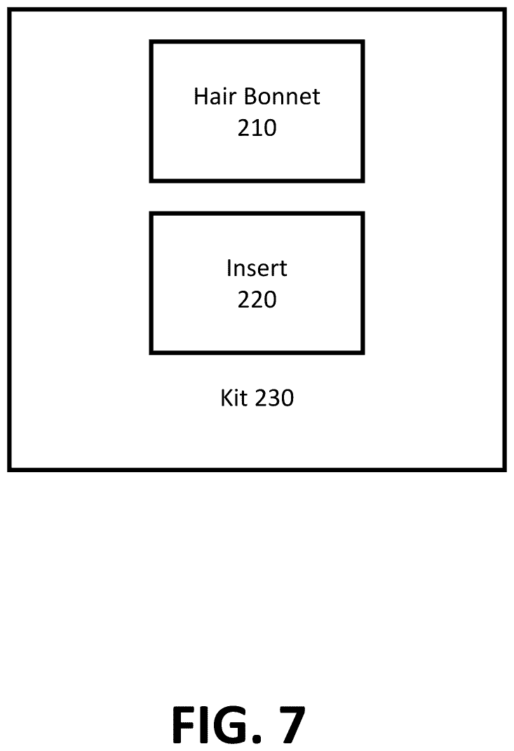

FIG. 7 is a block diagram of a kit 230 for a hair bonnet 210, according to some embodiments. The kit 230 includes a hair bonnet 210. The hair bonnet 210 may be any hair bonnet 10, 110 as described herein. Although not shown in FIG. 7, the hair bonnet 210 includes a protective hair covering made of fabric. The hair bonnet 210 also includes an eye mask coupled to the protective hair covering and the eye mask includes an opening. As shown in FIG. 7, the kit 230 also includes an insert 220 configured to be inserted into the opening of the eye mask. The insert 220 may be any insert 120 as described herein.

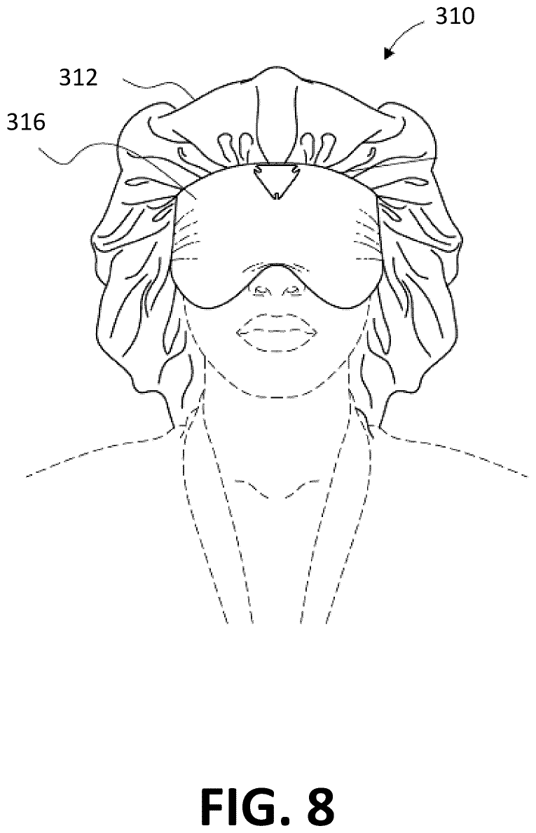

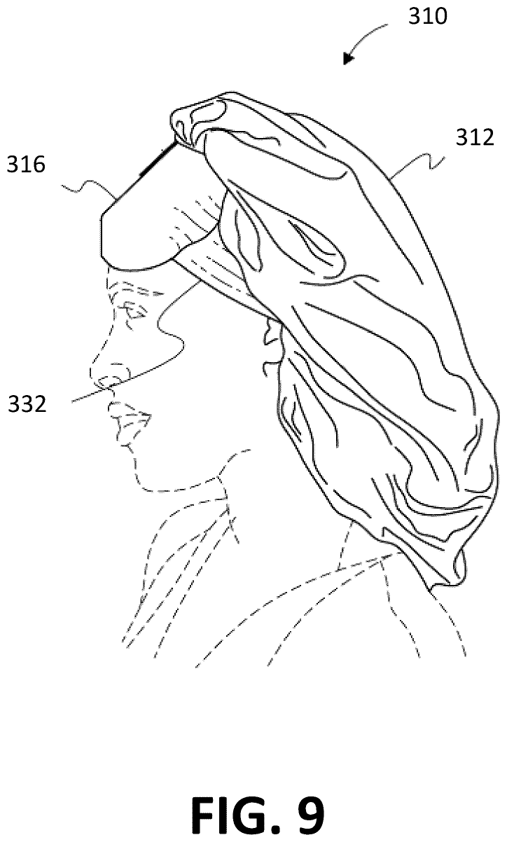

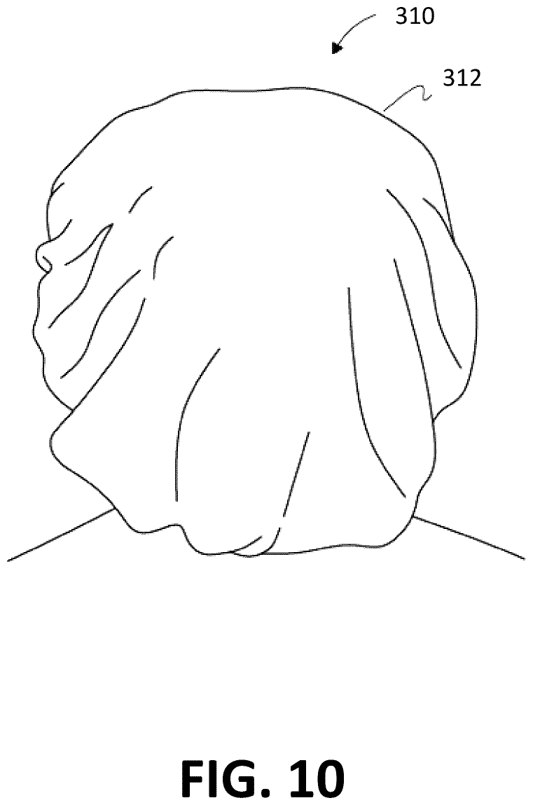

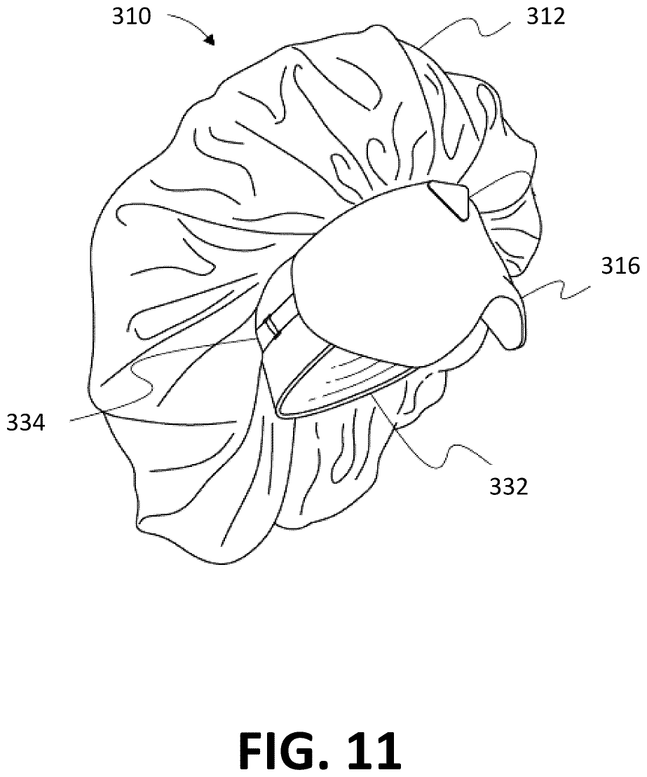

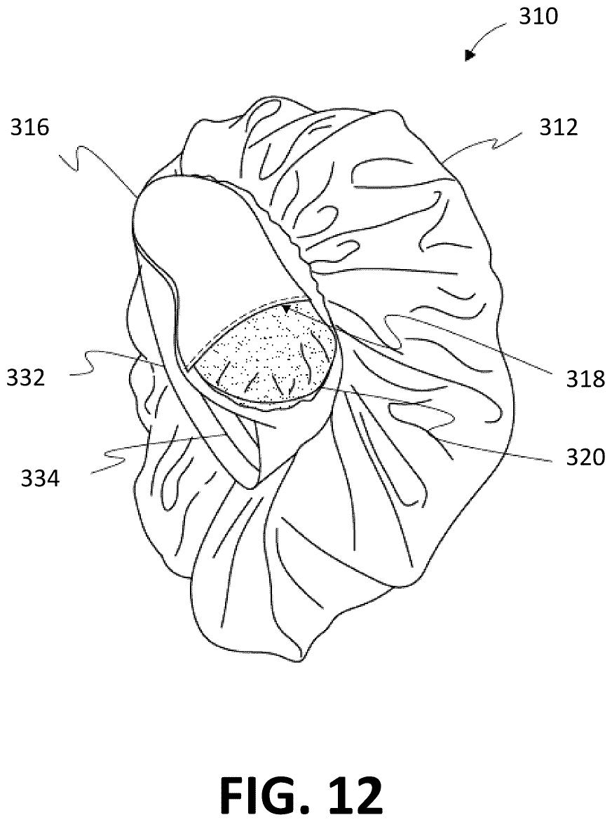

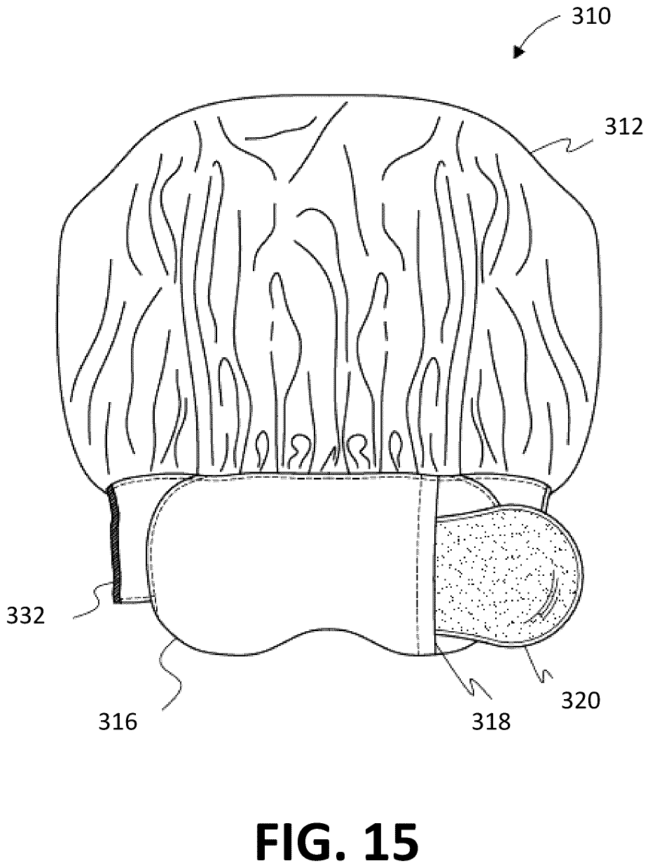

FIGS. 8-15 illustrate an alternative configuration for a hair bonnet 310 relative to the hair bonnets 10, 110, 210 of FIGS. 1-7. In FIGS. 8-15, the reference numerals are listed as 300-series reference numerals that represent structures and features that are similar to the structures and features of the hair bonnets 10, 110, 210 of FIGS. 1-7 that have two-digit, 100-series, or 200-series reference numerals, respectively.

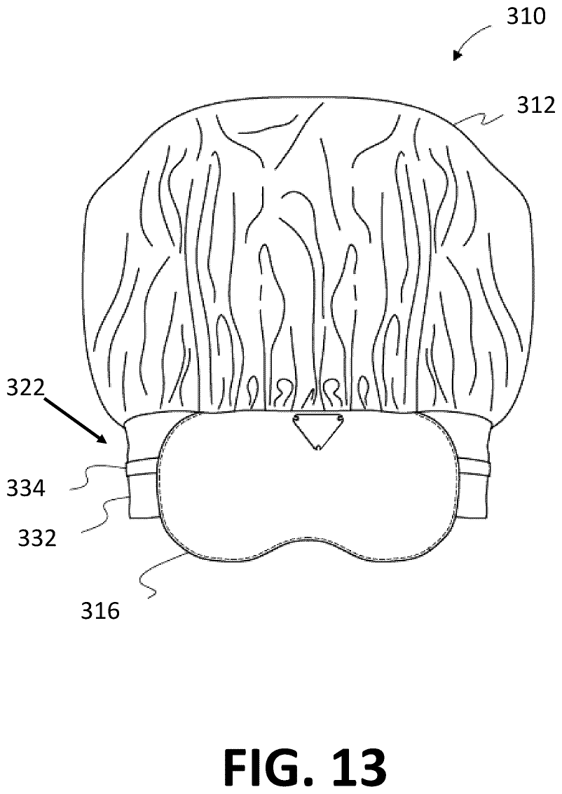

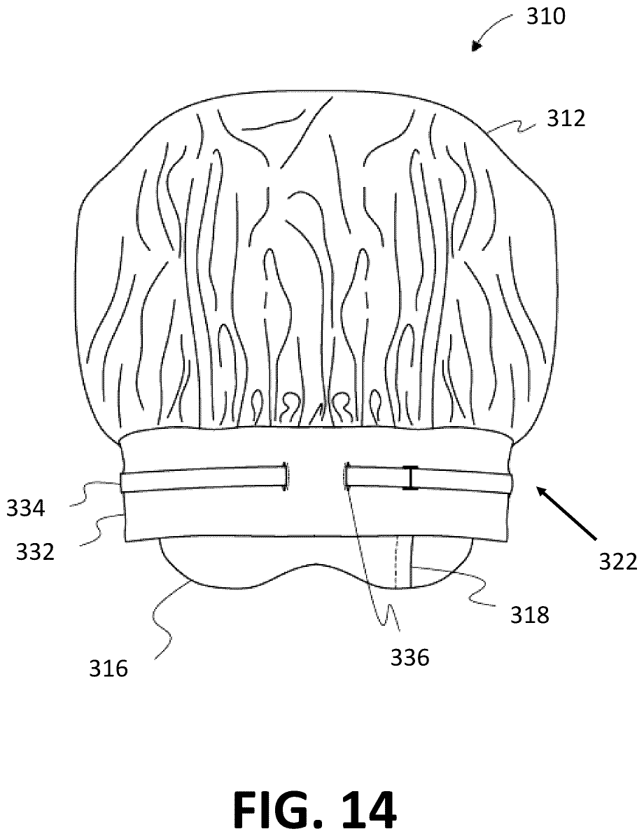

The hair bonnet 310 includes a protective hair covering 312 and an eye mask 316 coupled to the protective hair covering 312. As best shown in FIGS. 13-14, the securing mechanism 322 includes a headband 332 and an adjustable strap 334. In some embodiments, the headband 332 is made of flexible and/or stretchable fabric to fit snugly against a user's head. However, this is not required, and in some embodiments, the headband 332 is made of non-elastic material and the securing mechanism 332 relies primarily on the adjustable strap 334 to assist the hair bonnet 310 in fitting snugly against a user's head.

The adjustable strap 334 can be adjusted by any suitable means. For example, and in some embodiments, the adjustable strap 334 includes a flexible material, a ring, a slide, a hook, a tie down strap, a tension strap, and/or a buckle. As shown best in FIG. 14, the headband 332 includes a slit 336 through which the adjustable strap 334 extends. The slit 336 assists in keeping the adjustable strap 334 aligned with the headband 332.

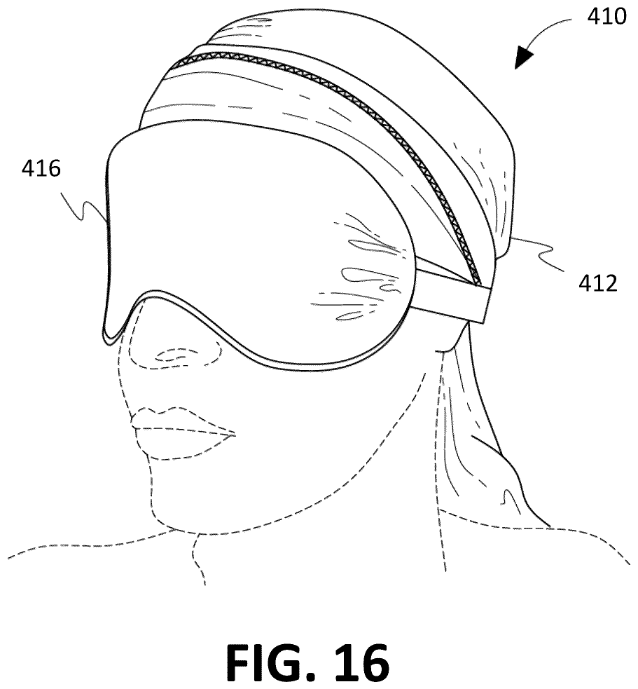

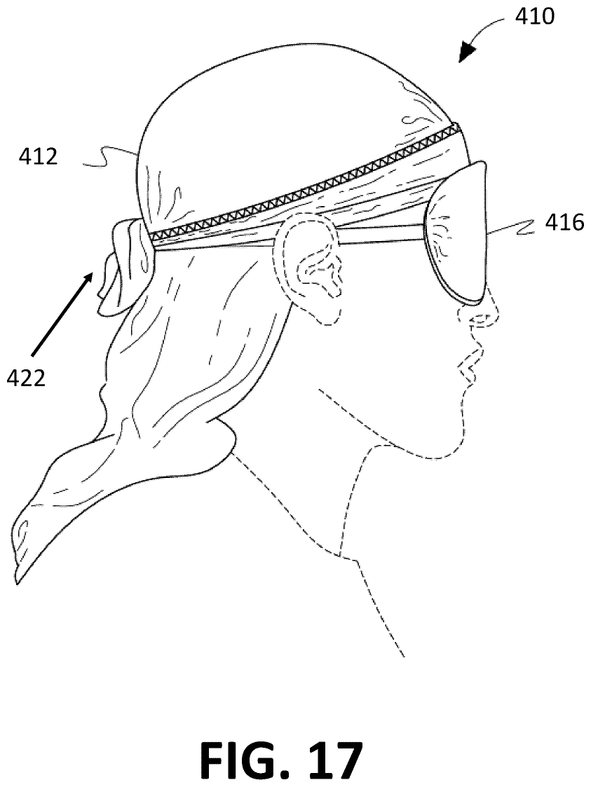

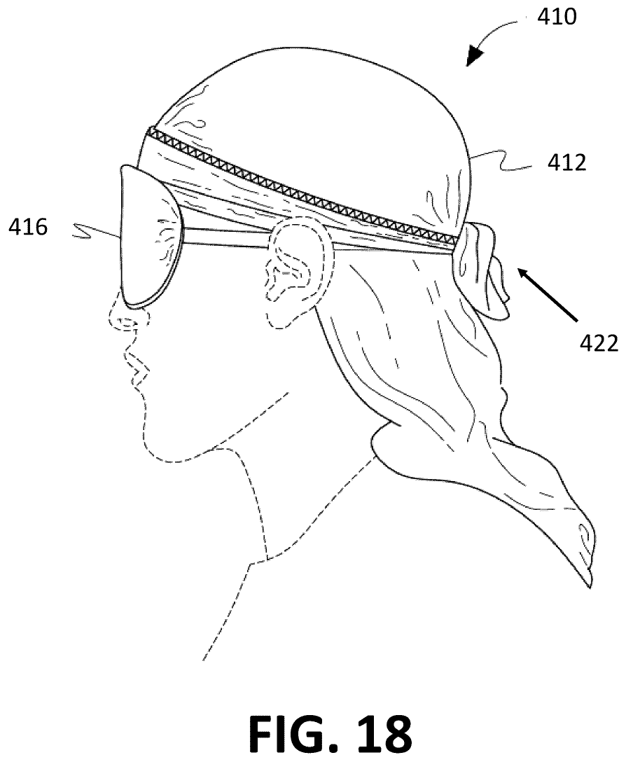

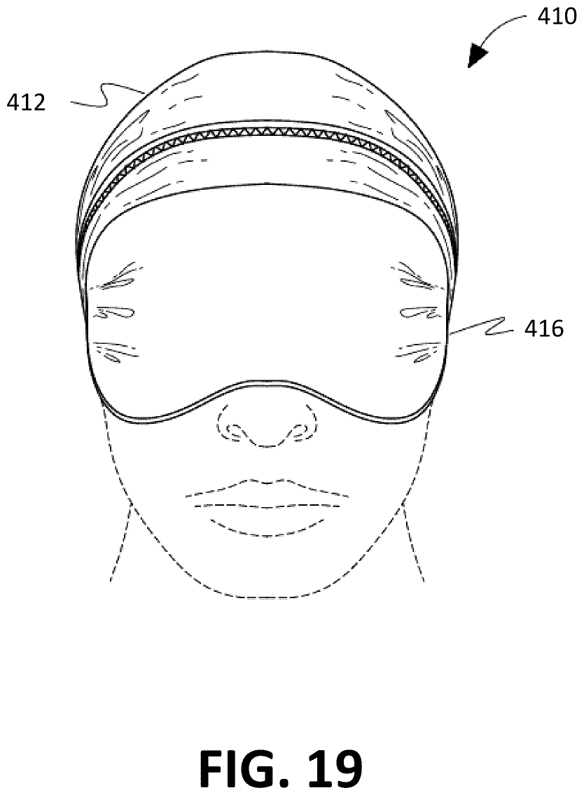

FIGS. 16-20 illustrate an alternative configuration for a hair bonnet 410 relative to the hair bonnets 10, 110, 210, 310 of FIGS. 1-15. In FIGS. 16-20, the reference numerals are listed as 400-series reference numerals that represent structures and features that are similar to the structures and features of the hair bonnets 10, 110, 210, 310 of FIGS. 1-15 that have two-digit, 100-series, 200-series, or 300-series reference numerals, respectively.

The hair bonnet 410 includes a protective hair covering 412 and an eye mask 416 coupled to the protective hair covering 412. The protective hair covering 412 is shaped and configured to be worn like a durag. For example, the tighter and compressed fit of the protective hair covering 412 assists in maintaining waves in hair, maintaining braids in hair, and/or keeping hair closer to the head.

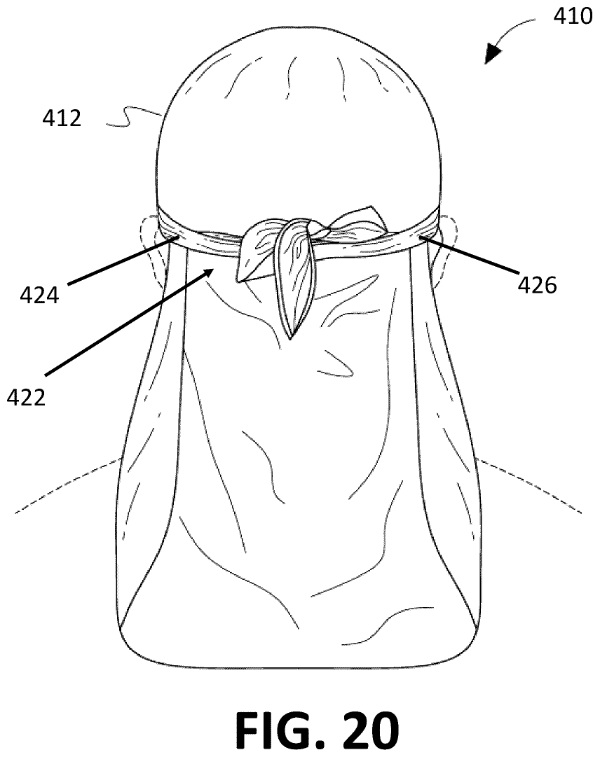

As shown best in FIG. 20, the securing mechanism 422 includes a first strap 424 and a second strap 426. The first strap 424 and the second strap 426 are tied together to assist in keeping the hair bonnet 410 in place on a user's head. For example, the first strap 424 and the second strap 426 can be tied at the back of a user's head to keep the hair bonnet 410 in place while the user sleeps.

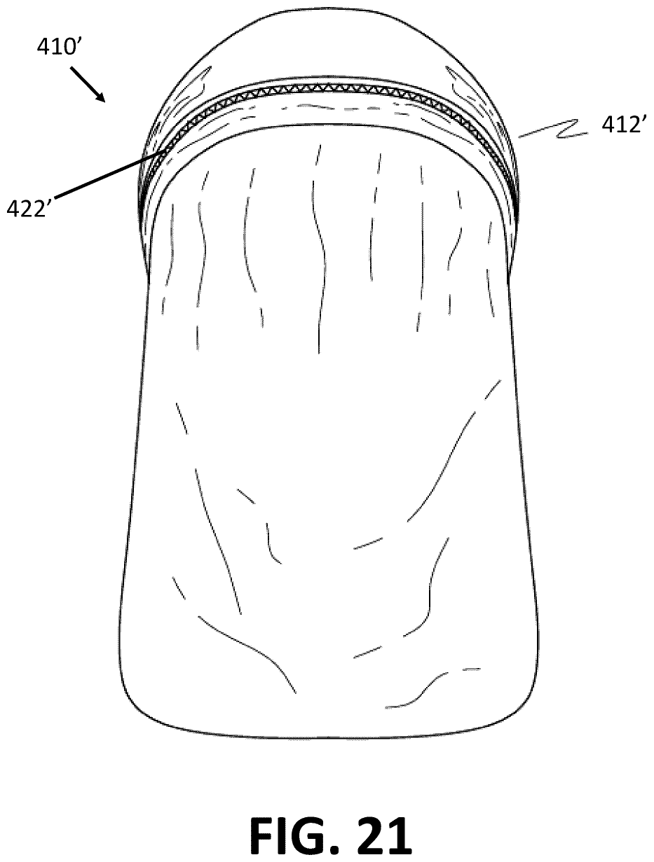

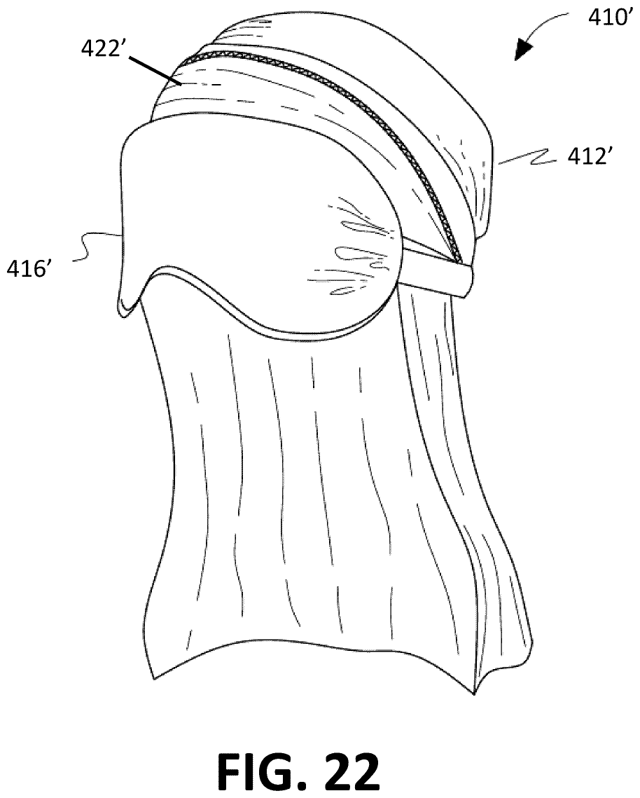

FIGS. 21-22 illustrate an alternative configuration for a hair bonnet 410′ relative to the hair bonnet 10, 110, 210, 310, 410 of FIGS. 1-20. In FIGS. 21-22, the reference numerals are listed as 400′-series reference numerals that represent structures and features that are similar to the structures and features of the hair bonnets 10, 110, 210, 310, 410 of FIGS. 1-20 that have two-digit, 100-series, 200-series, 300-series, or 400-series reference numerals, respectively.

The hair bonnet 410′ includes a protective hair covering 412′ and an eye mask 416′ coupled to the protective hair covering 412′. Similar to FIGS. 16-20, the protective hair covering 412′ is shaped and configured to be worn like a durag. However, the securing mechanism 422′ is elastic which assists the hair bonnet 410′ in snuggly fitting against a user's head.

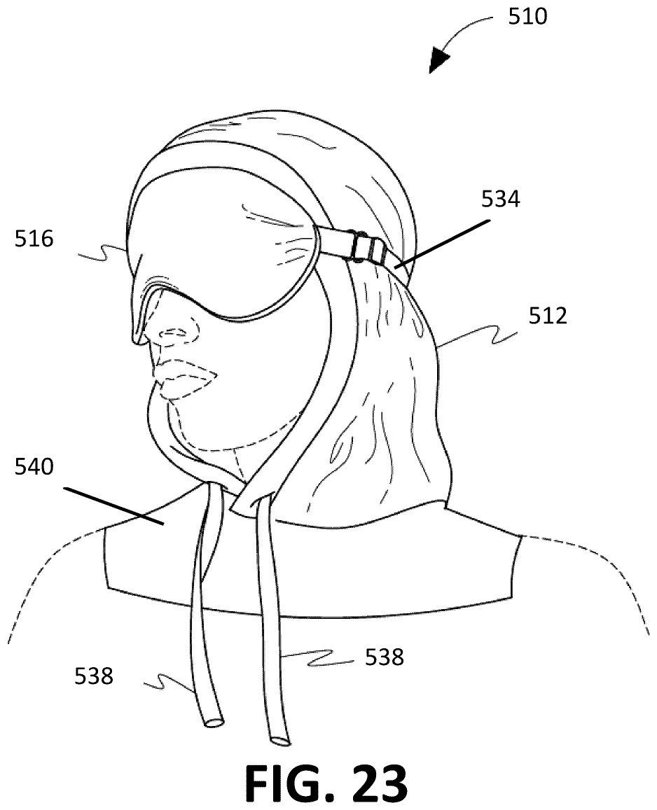

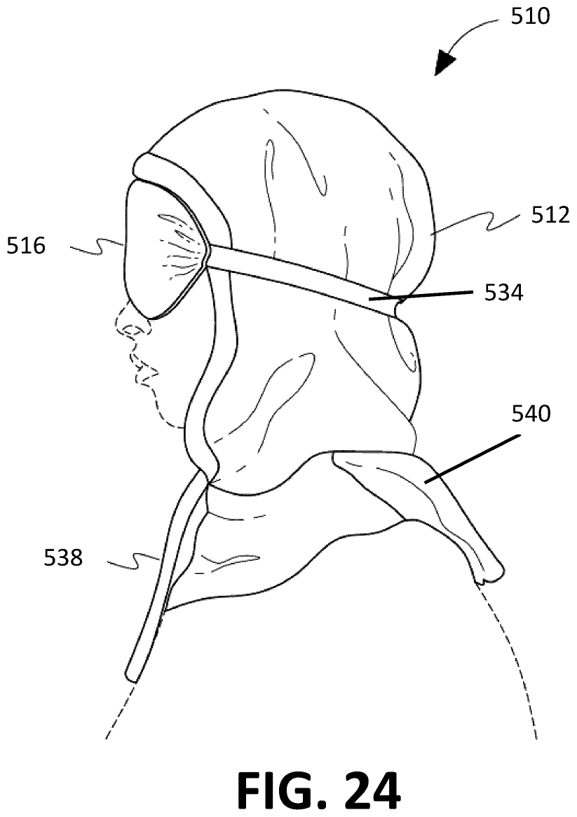

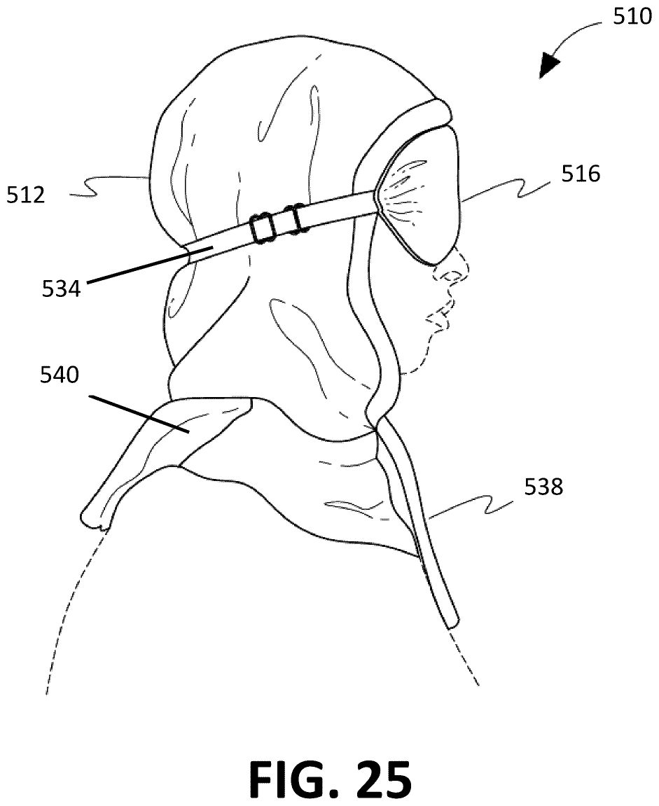

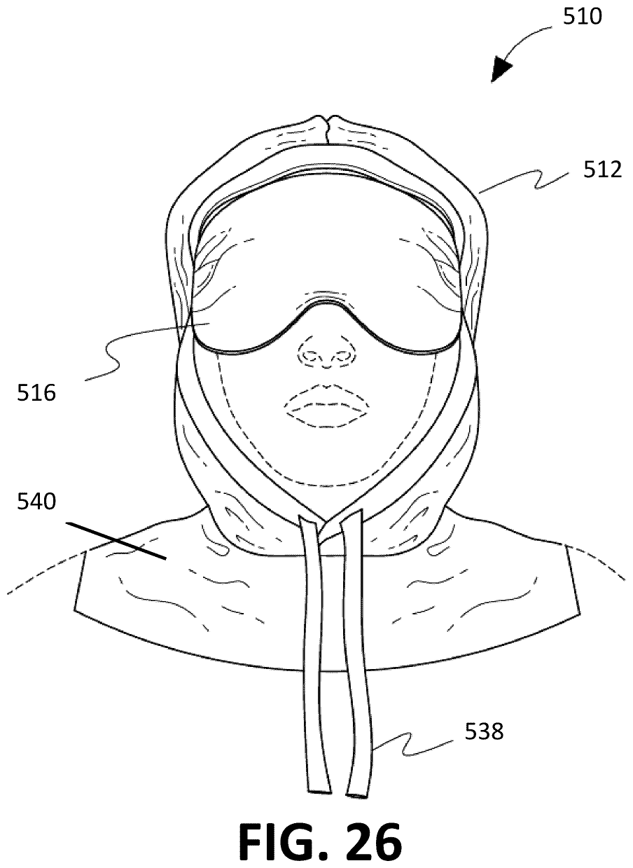

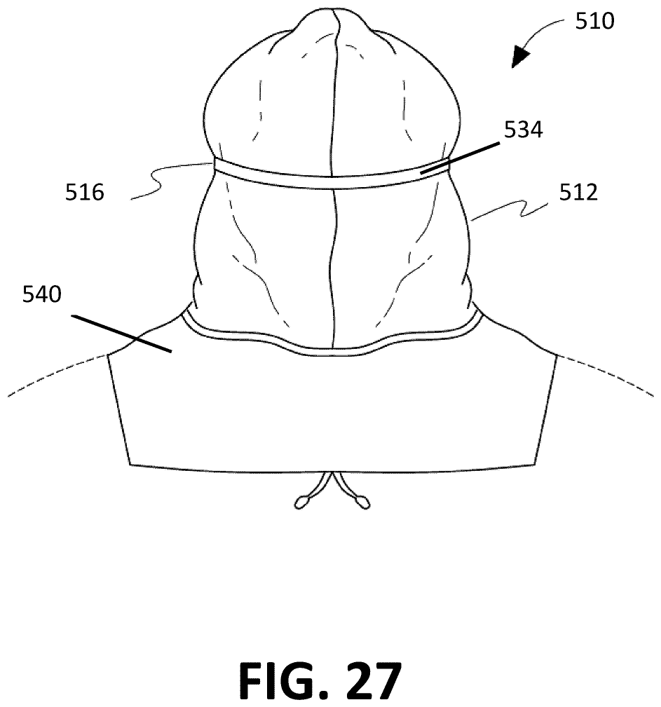

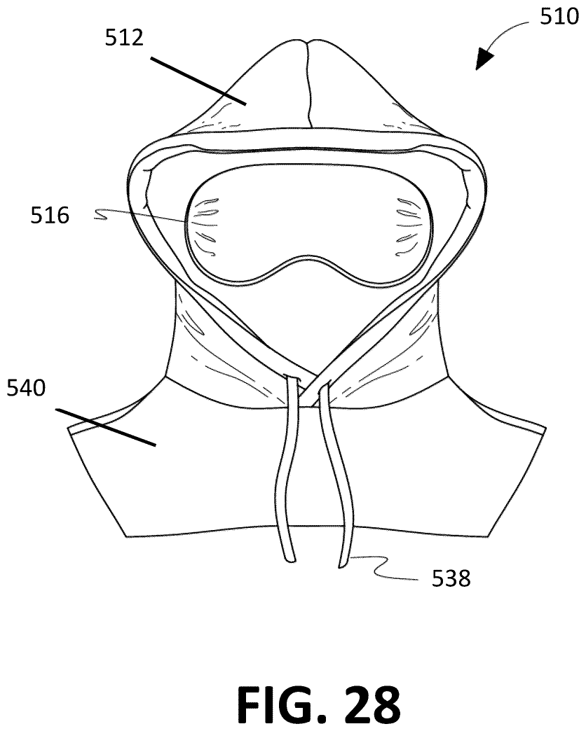

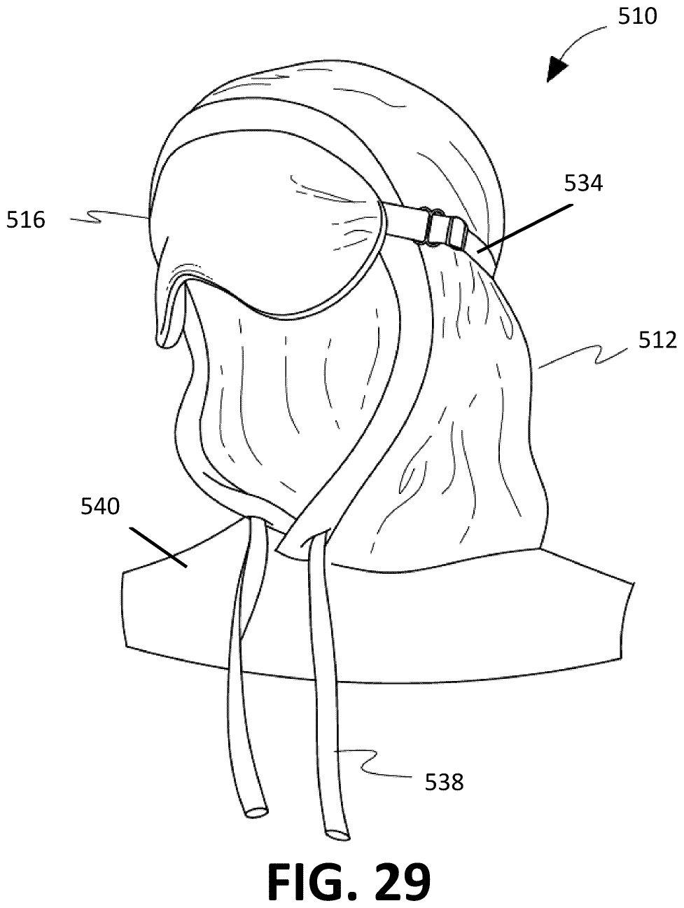

FIGS. 23-29 illustrate an alternative configuration for a hair bonnet 510 relative to the hair bonnets 10, 110, 210, 310, 410, 410′ of FIGS. 1-22. In FIGS. 23-29, the reference numerals are listed as 500-series reference numerals that represent structures and features that are similar to the structures and features of the hair bonnets 10, 110, 210, 310, 410, 410′ of FIGS. 1-22 that have two-digit, 100-series, 200-series, 300-series, 400-series, or 400′-series reference numerals, respectively.

The hair bonnet 510 includes a protective hair covering 512 and an eye mask 516 coupled to the protective hair covering 512. The protective hair covering 512 is shaped and configured to be worn as a hood portion of a hoodie. The protective hair covering 512 is coupled to a sweatshirt 540, for example, to assist the protective hair covering 512 in remaining on a head of a user. In some embodiments, the sweatshirt 540 is made of the same material as the protective hair covering 512. However, this is not required, and in some embodiments the sweatshirt 540 is made of a different material or different combination of materials than the protective hair covering 512. The sweatshirt 540 is made of any suitable material. For example, and in some embodiments, the sweatshirt 540 is made of cotton, fleece, polyester, wool, satin, acrylic, silk, bamboo fabric, and/or French Terry.

The hair bonnet 510 also includes a drawstring 538 that extends through a casing at an edge of the protective hair covering 512. The drawstring 538 assists in keeping the hair bonnet 510 near a user's head. For example, when the drawstring 538 is pulled, the edge of the protective hair covering 512 gathers around the user's face and assists in encasing the user's hair within the protective hair covering 512.

The hair bonnet 510 further includes an adjustable strap 534 that assists in tightening and/or loosening the eye mask 516 on the user's head. The adjustable strap 534 can be adjusted by any suitable means. For example, and in some embodiments, the adjustable strap 534 includes a flexible material, a ring, a slide, a hook, a tie down strap, a tension strap, and/or a buckle.

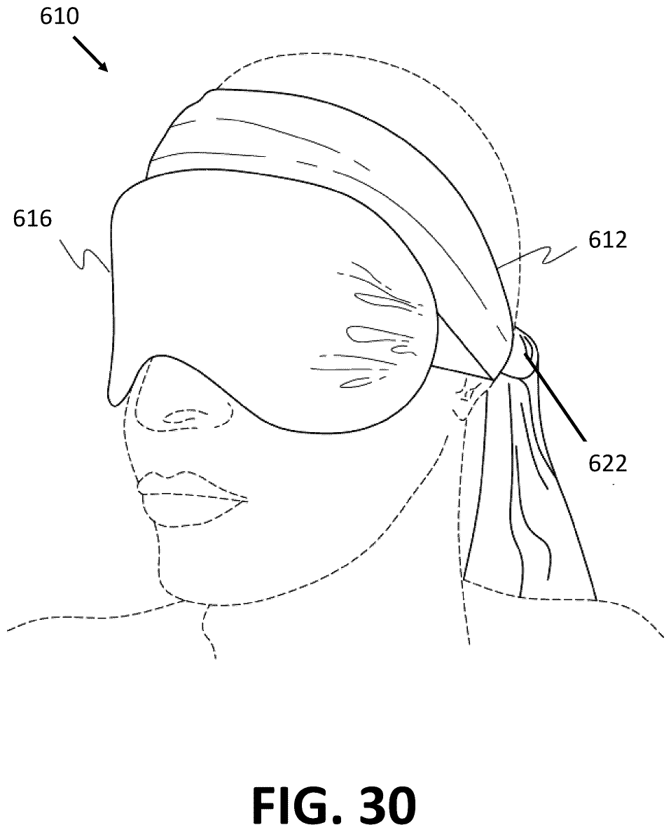

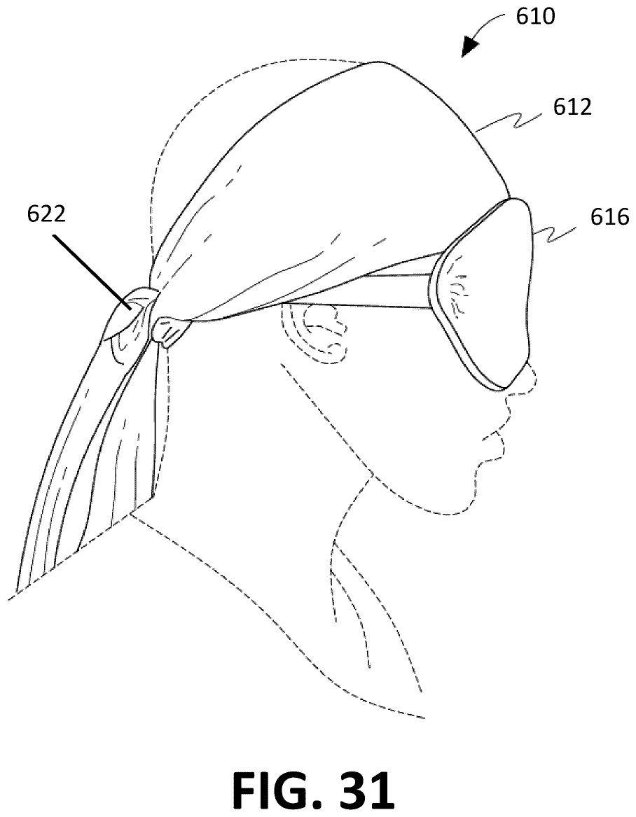

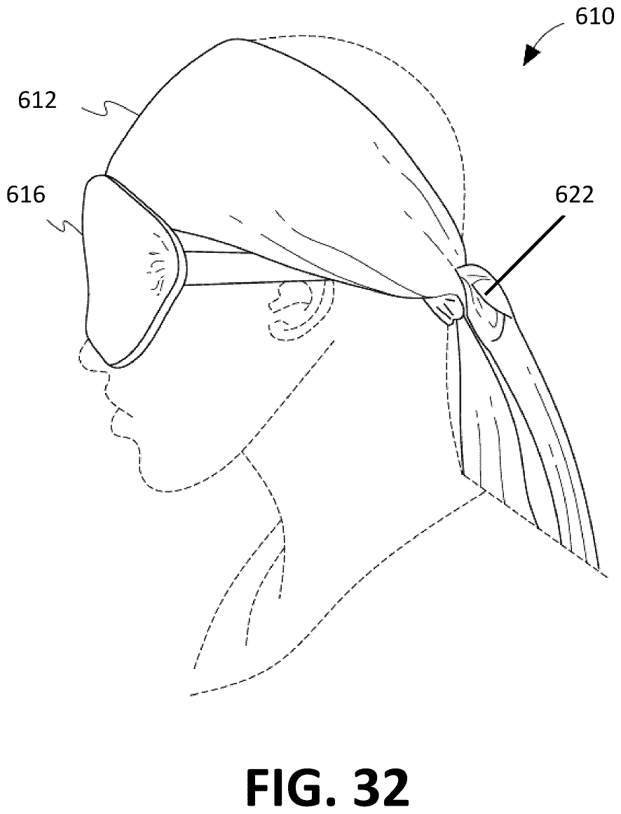

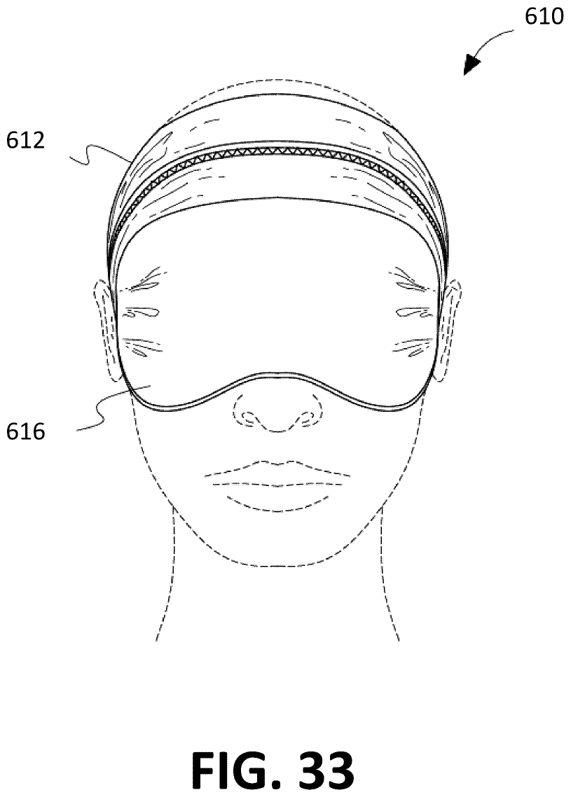

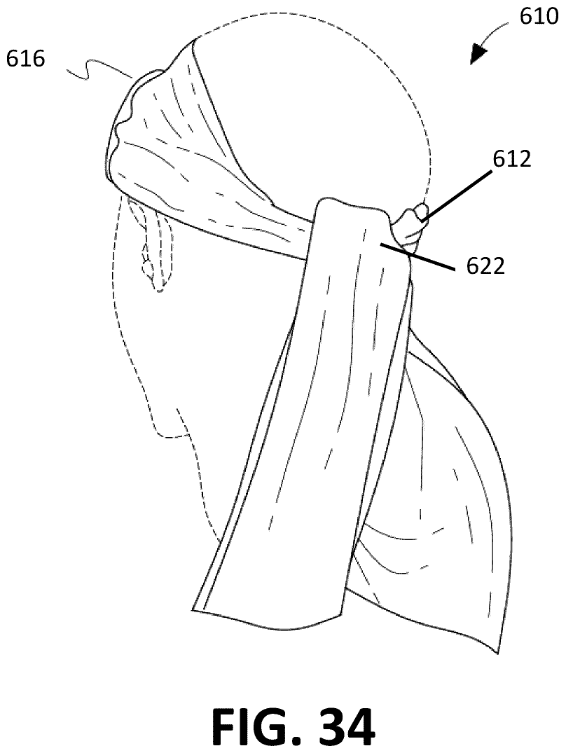

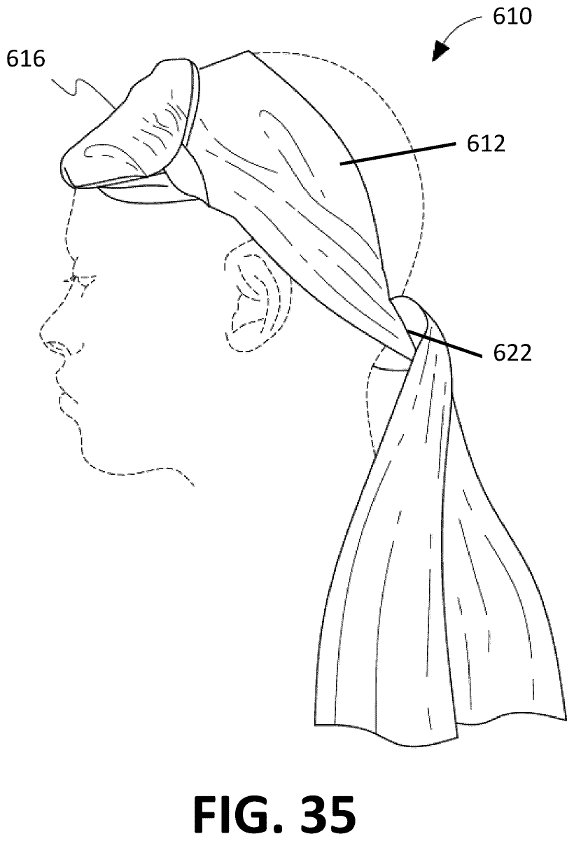

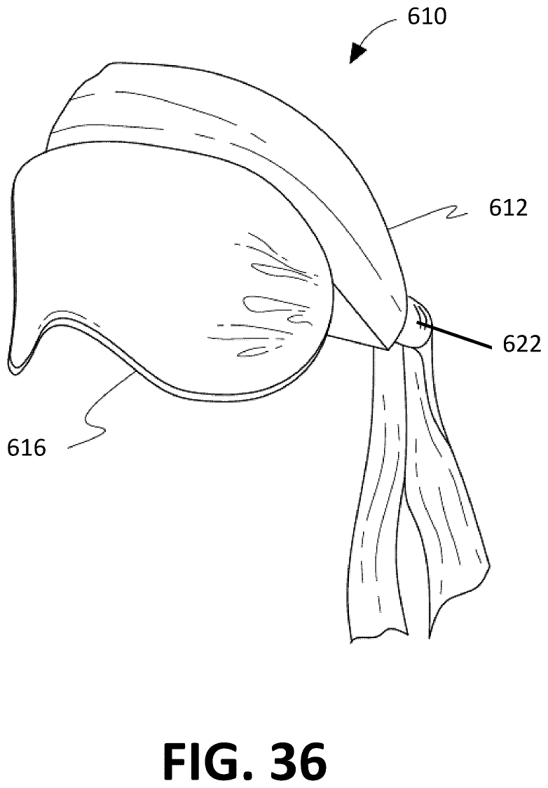

FIGS. 30-36 illustrate an alternative configuration for a hair bonnet 610 relative to the hair bonnets 10, 110, 210, 310, 410, 410′, 510 of FIGS. 1-29. In FIGS. 30-36, the reference numerals are listed as 600-series reference numerals that represent structures and features that are similar to the structures and features of the hair bonnets 10, 110, 210, 310, 410, 410′, 510 of FIGS. 1-29 that have two-digit, 100-series, 200-series, 300-series, 400-series, 400′-series, or 500-series reference numerals, respectively.

The hair bonnet 610 includes a protective hair covering 612 and an eye mask 616 coupled to the protective hair covering 612. The protective hair covering 612 is shaped and configured similar to a scarf. The protective hair covering 612 is configured to wrap around a user's head and is secured to the user's head via a securing mechanism 622. The securing mechanism 622 is a tie between two ends of the protective hair covering 612. However, this is not required, and in some embodiments, the securing mechanism 622 includes snaps, buttons and holes, hook and loop fasteners, G-hooks and loops, S-hooks and loops, or any other suitable fasteners to secure the protective hair covering 612 on a user's head.

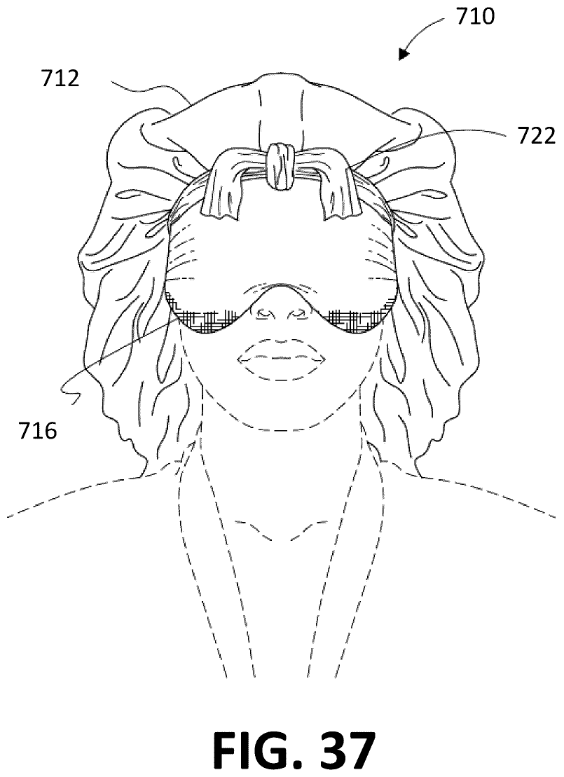

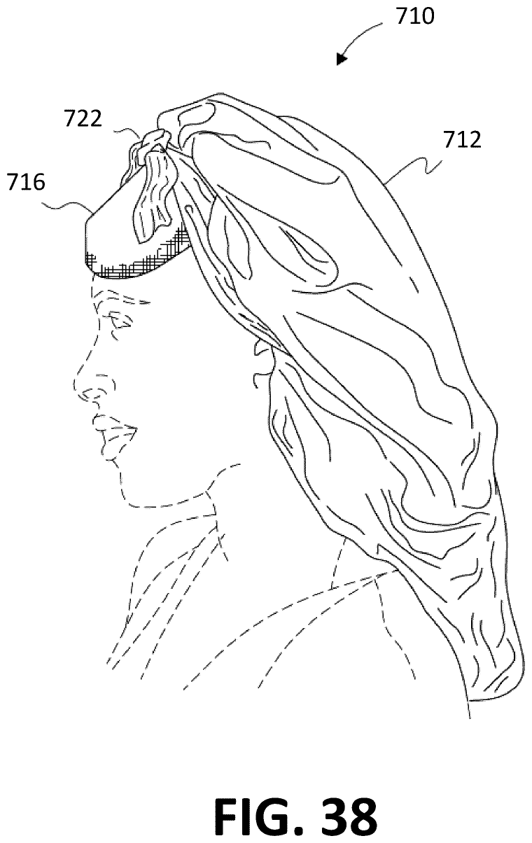

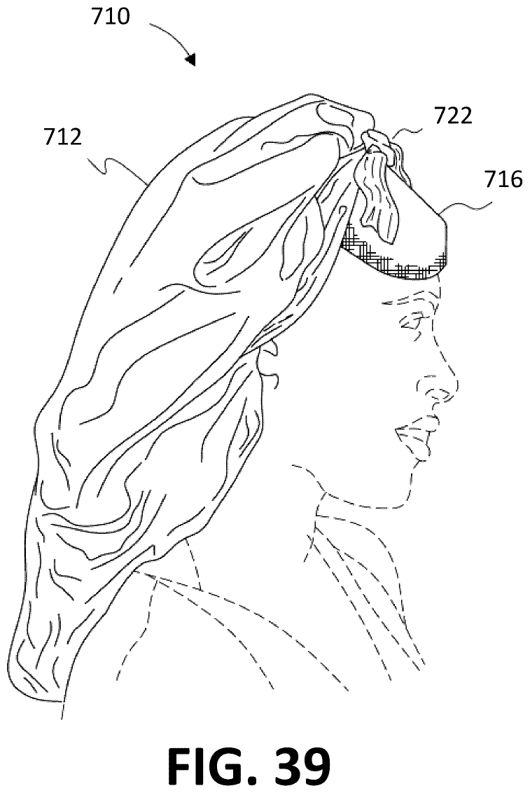

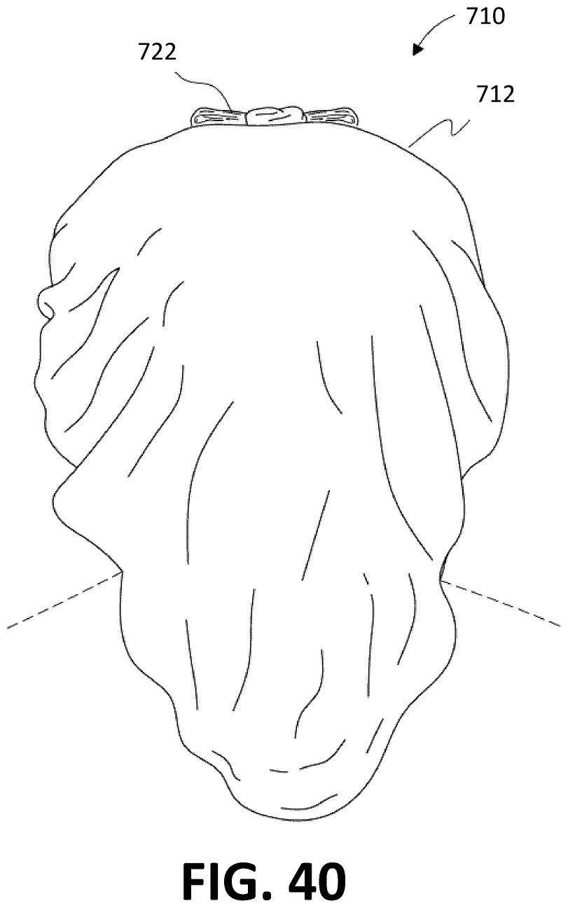

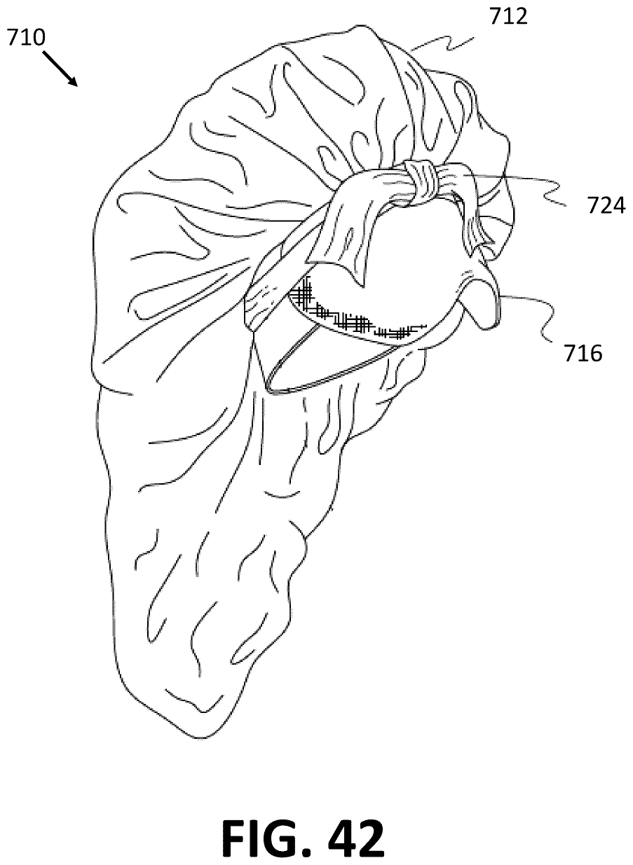

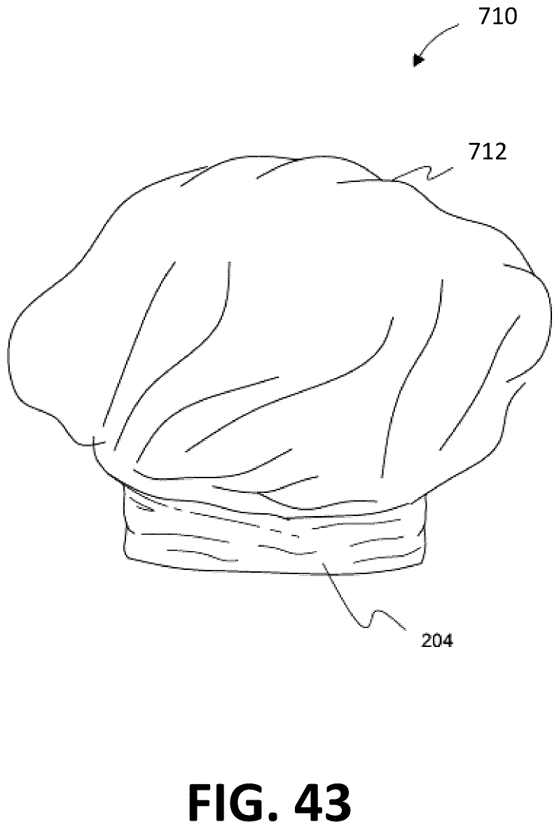

FIGS. 37-43 illustrate an alternative configuration for a hair bonnet 710 relative to the hair bonnets 10, 110, 210, 310, 410, 410′, 510, 610 of FIGS. 1-36. In FIGS. 37-43, the reference numerals are listed as 700-series reference numerals that represent structures and features that are similar to the structures and features of the hair bonnets 10, 110, 210, 310, 410, 410′, 510, 610 of FIGS. 1-36 that have two-digit, 100-series, 200-series, 300-series, 400-series, 400′-series, 500-series, or 600-series reference numerals, respectively.

The hair bonnet 710 includes a protective hair covering 712 and an eye mask 716 coupled to the protective hair covering 712. The protective hair covering 712 extends past the shoulders of the user to assist in enveloping long hair. For example, the long length of the protective hair covering 712 assists in preventing longer hair from being scrunched inside of the protective hair covering 712.

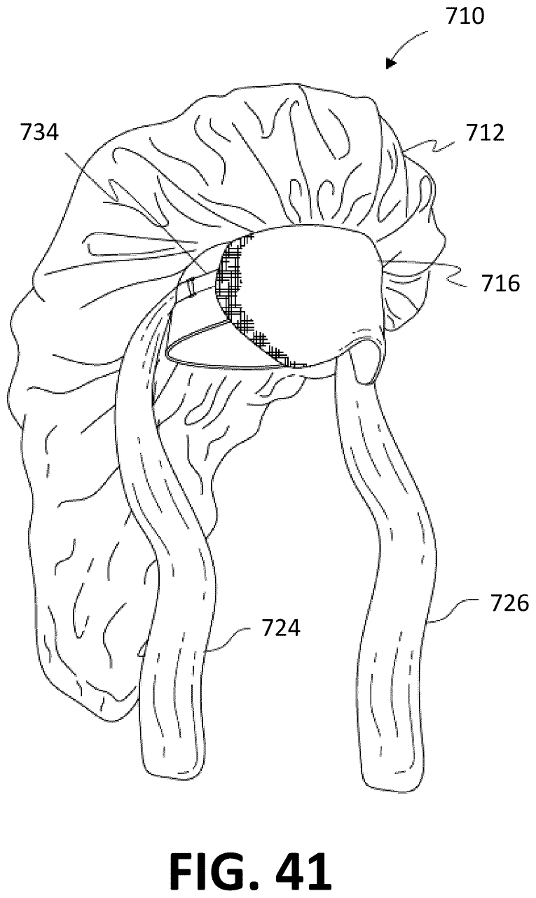

The hair bonnet 710 further includes a securing mechanism 722. As best shown in FIG. 41, the securing mechanism 722 includes a first strap 724 and a second strap 726. In some embodiments, the first strap 724 and/or the second strap 726 comprise a same material as that of the protective hair covering 712. In some embodiments, the first strap 724 and/or the second strap 726 are made of fabric. Examples of suitable fabrics include, but are not limited to, satin, polyester, acrylic, silk, satin, and/or bamboo fabric. In some embodiments, the first strap 724 and/or the second strap 726 additionally include elastic. In some embodiments, the first strap 724 and the second strap 726 are made of the same material. However, this is not required and in other embodiments, the first strap 724 and the second strap 726 are made of different material.

As best shown in FIGS. 37-39, the first strap 724 and the second strap 726 are configured to wrap around a head of a user and tie together to securely hold the hair bonnet 710 on a user's head. For example, and in some embodiments, the first strap 724 and the second strap 726 extend from the hair bonnet 710 at the nape of the user's neck. To secure the hair bonnet 710, the first strap 724 and the second strap 726 may be pulled around the crown of the user's head and be tied together (e.g., in a bow) above the eye mask 716 (e.g., as shown in FIG. 37). In another embodiment, the first strap 724 and the second strap 726 may wrap around the forehead of the user and be tied together (e.g., in a bow) at the nape of the user's neck.

As shown in FIG. 41, the hair bonnet 710 also includes an adjustable strap 734 that assists in tightening and/or loosening the eye mask 716 on the user's head. The adjustable strap 734 can be adjusted by any suitable means. For example, and in some embodiments, the adjustable strap 734 includes a flexible material, a ring, a slide, a hook, a tie down strap, a tension strap, and/or a buckle.

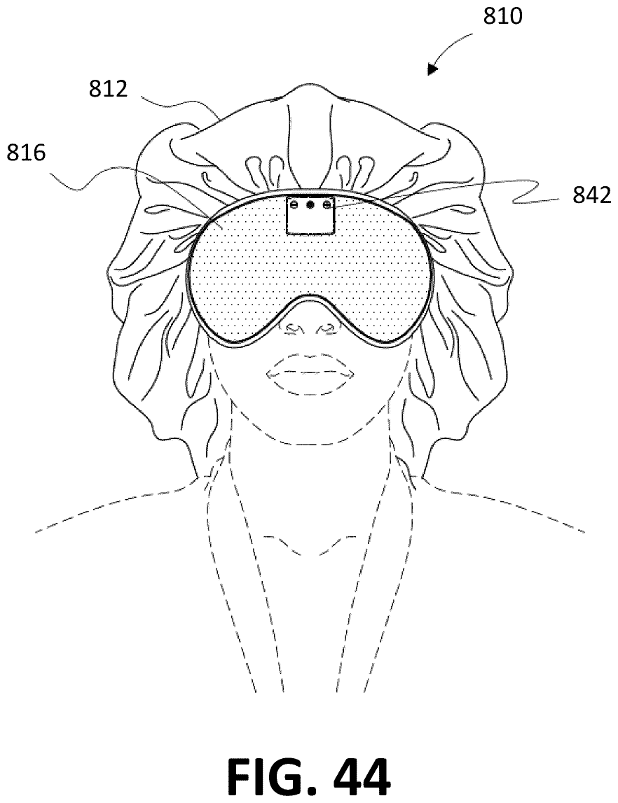

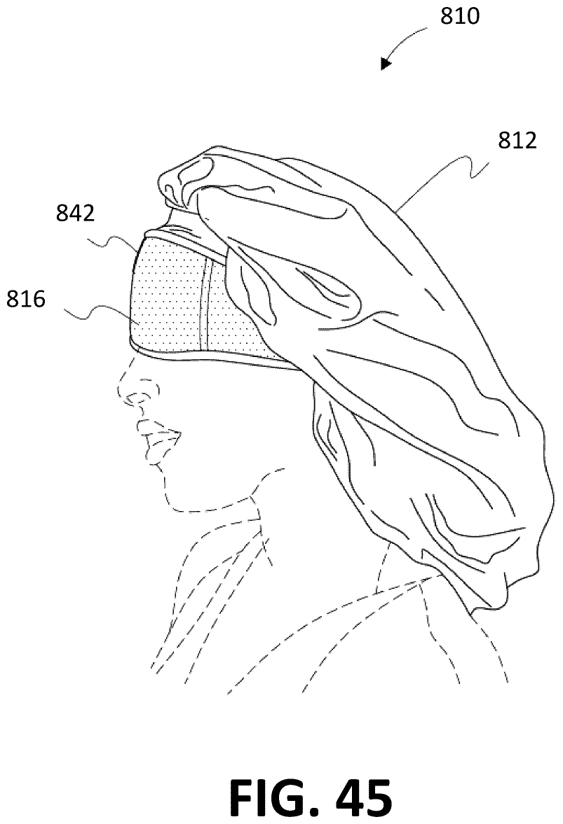

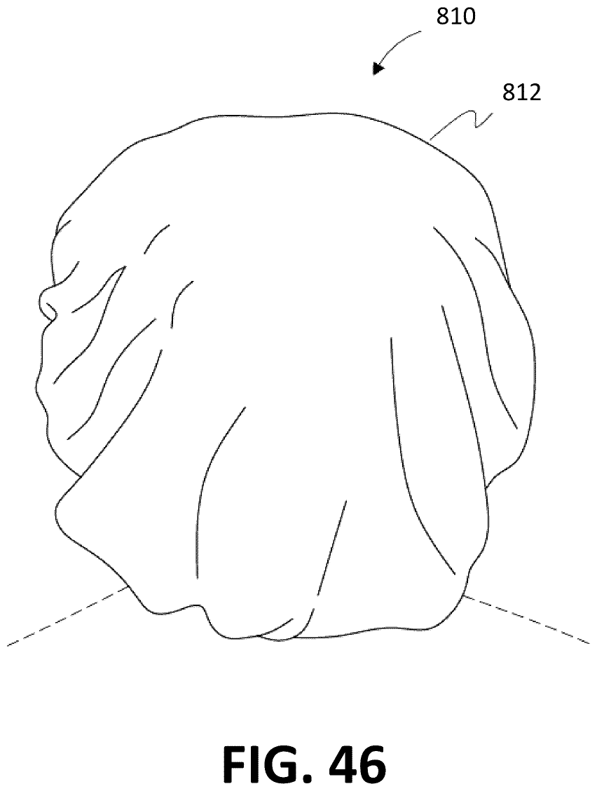

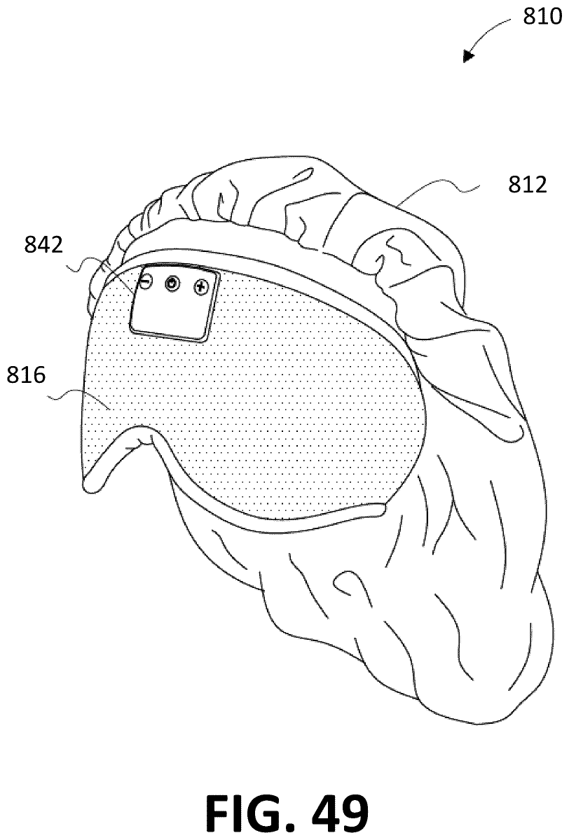

FIGS. 44-50 illustrate an alternative configuration for a hair bonnet 810 relative to the hair bonnets 10, 110, 210, 310, 410, 410′, 510, 610, 710 of FIGS. 1-43. In FIGS. 44-50, the reference numerals are listed as 800-series reference numerals that represent structures and features that are similar to the structures and features of the hair bonnets 10, 110, 210, 310, 410, 410′, 510, 610, 710 of FIGS. 1-43 that have two-digit, 100-series, 200-series, 300-series, 400-series, 400′-series, 500-series, 600-series, or 700-series reference numerals, respectively.

The hair bonnet 810 includes a protective hair covering 812 and an eye mask 816 coupled to the protective hair covering 812. The hair bonnet 810 further includes a control panel 842 that allows the user to control audio that the user is listening to. For example, and in some embodiments, the control panel 842 includes pause, play, volume increase, volume decrease, power on/off, skip, and/or repeat buttons. In some embodiments, the control panel 842 is communicatively coupled to a user device, such as a smartphone, via radio waves, Bluetooth, WiFi, a wire, or any other suitable means. For example, the user device can be used to select and/or play a movie, a show, a song, and/or other audio.

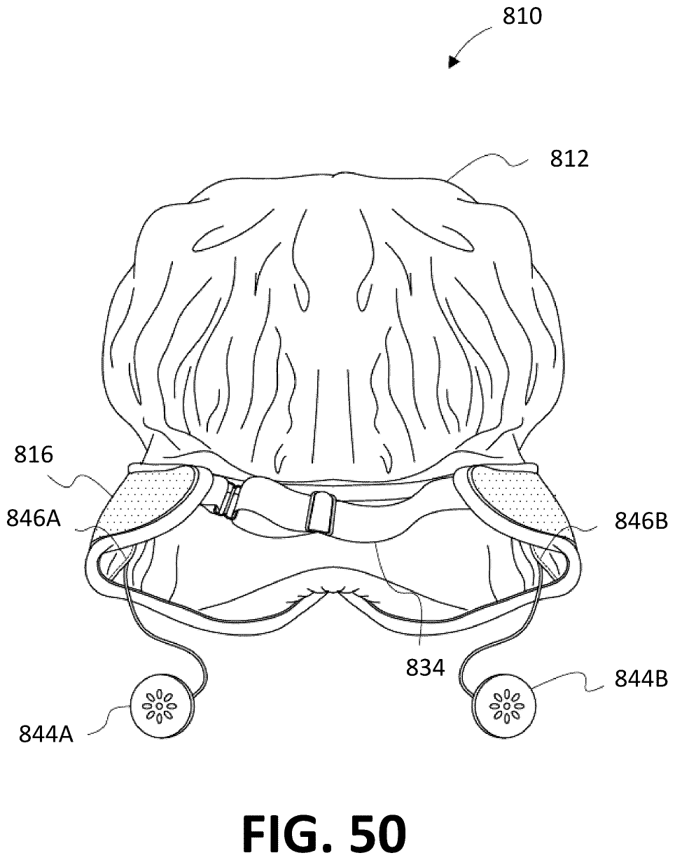

The user may listen to the audio via speakers 844A, 844B, shown in FIG. 50. The speakers 844A, 844B are connected to the control panel 842 via a wire. However, this is not required, and in some embodiments, the speakers 844A, 844B are connected to the control panel 842 wirelessly, such as via radio waves, Bluetooth, WiFi, or any other suitable means. The hair bonnet 810 also includes speaker pockets 846A, 846B into which the speakers 844A, 844B can be placed. The speaker pockets 846A, 846B are shaped and positioned to align with ears of the user.

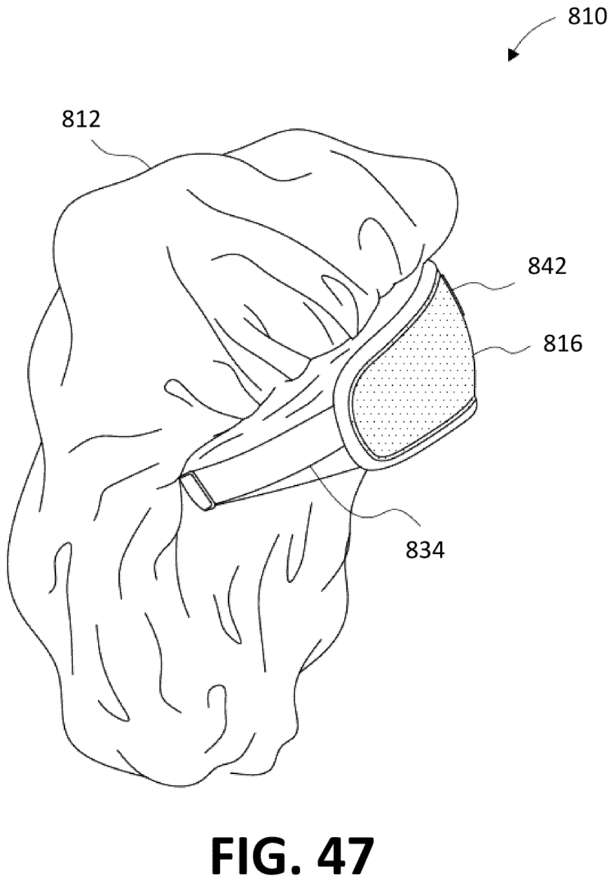

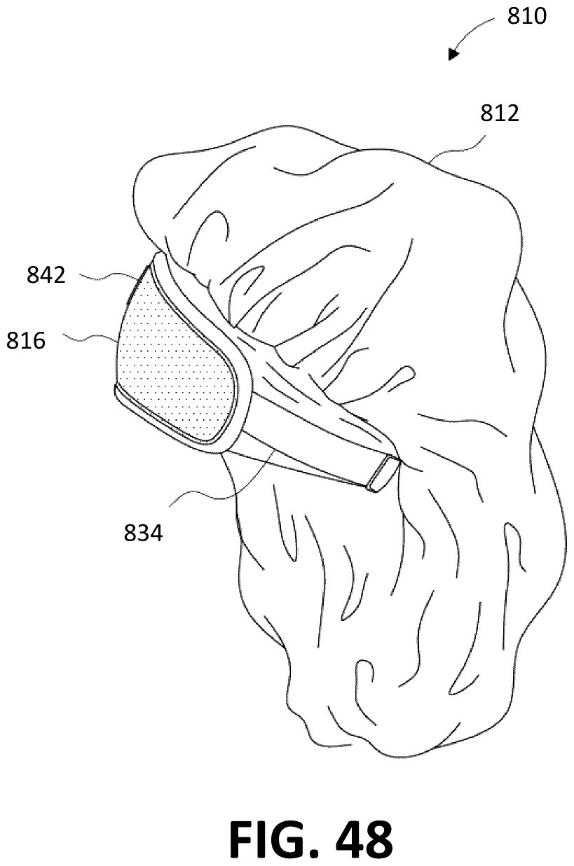

As shown in FIGS. 47 and 48, the hair bonnet 810 also includes an adjustable strap 834 that assists in tightening and/or loosening the eye mask 816 on the user's head. The adjustable strap 834 can be adjusted by any suitable means. For example, and in some embodiments, the adjustable strap 834 includes a flexible material, a ring, a slide, a hook, a tie down strap, a tension strap, and/or a buckle.

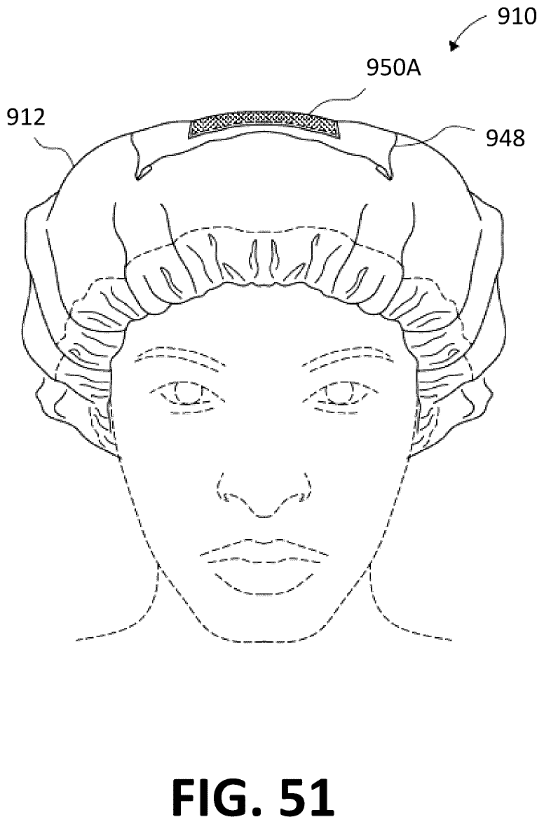

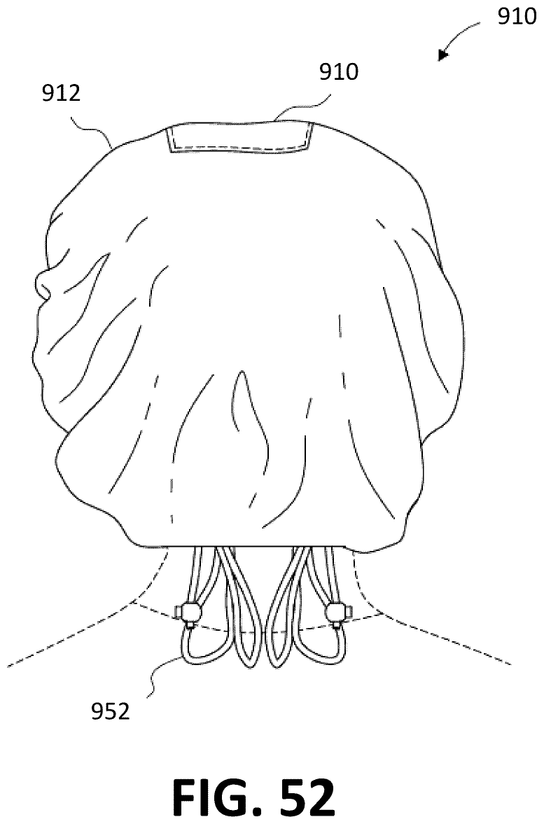

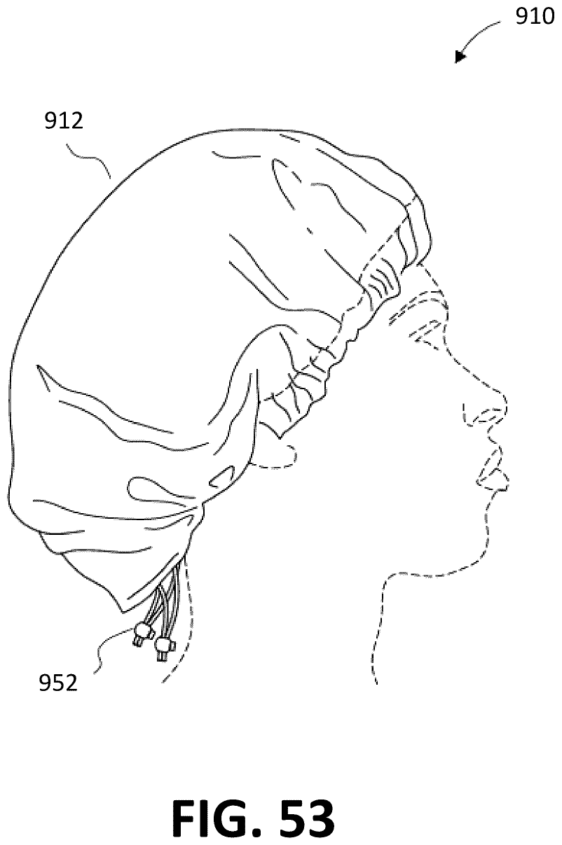

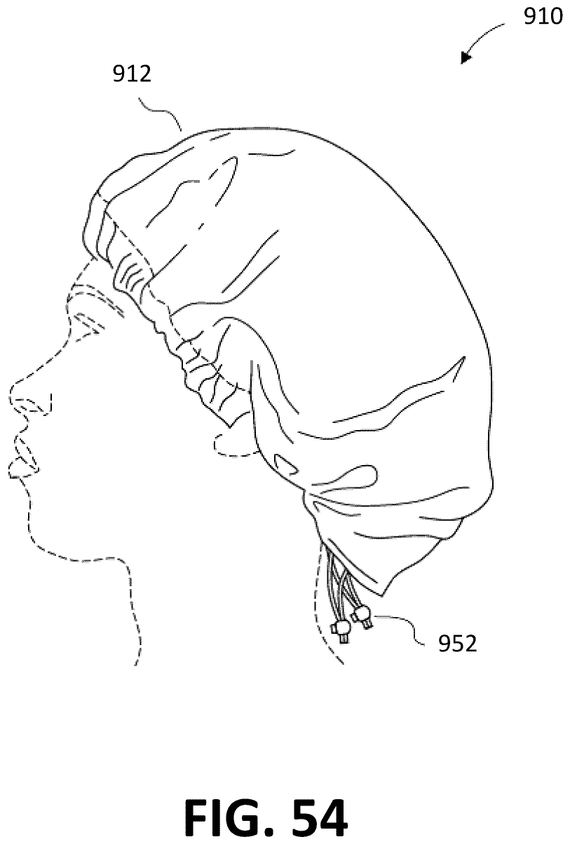

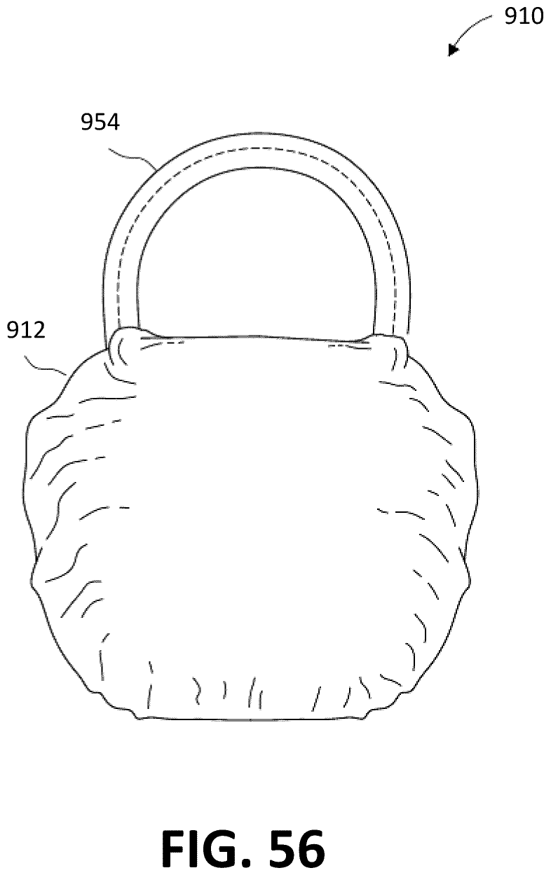

FIGS. 51-57 illustrate an alternative configuration for a hair bonnet 910 relative to the hair bonnets 10, 110, 210, 310, 410, 410′, 510, 610, 710, 810 of FIGS. 1-50. In FIGS. 51-57, the reference numerals are listed as 900-series reference numerals that represent structures and features that are similar to the structures and features of the hair bonnets 10, 110, 210, 310, 410, 410′, 510, 610, 710, 810 of FIGS. 1-50 that have two-digit, 100-series, 200-series, 300-series, 400-series, 400′-series, 500-series, 600-series, 700-series, or 800-series reference numerals, respectively.

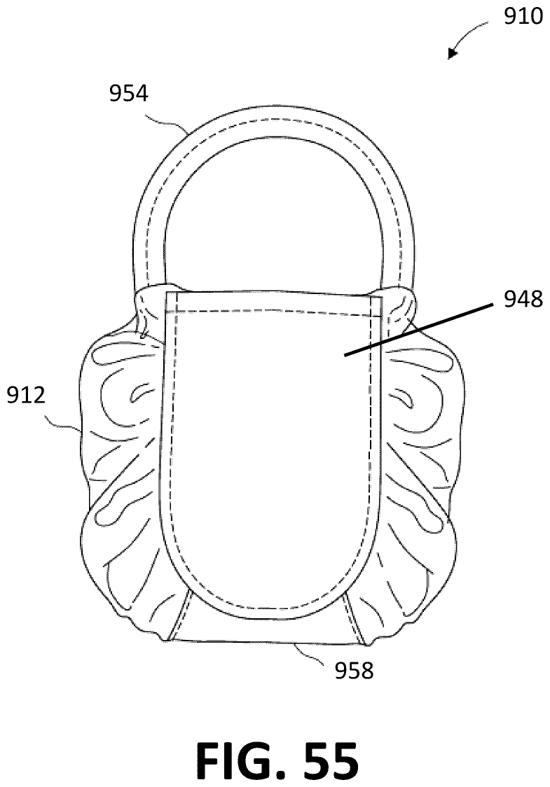

The hair bonnet 910 includes a protective hair covering 912 and an eye mask 916 coupled to the protective hair covering 912. As shown best in FIG. 55, the hair bonnet 910 is configured to fold into the shape of a makeup bag and/or handbag. The hair bonnet 910 includes a handle 954 by which a user can carry the hair bonnet 910 while it is folded into a bag shape. The hair bonnet 910 further includes a pocket 948 where a user can store additional items such as makeup, credit cards, tissues, combs, brushes, etc.

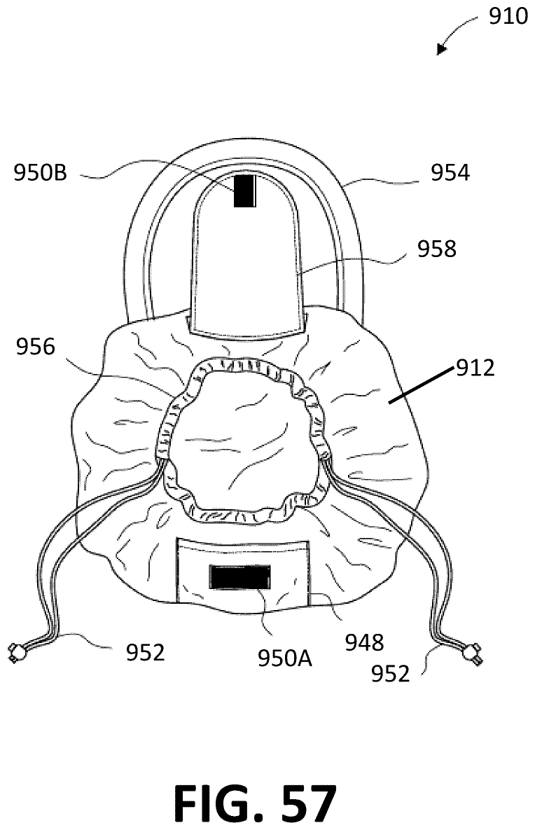

As best shown in FIG. 57, the hair bonnet 910 includes adjustable cords 952 that extend through a casing 956 that extends along the edge of the protective hair covering 912. When the hair bonnet 910 is used as a hair bonnet, the adjustable cords 952 assist in keeping the hair bonnet 910 near a user's head. For example, when the adjustable cords 952 are pulled, the edge of the protective hair covering 912 gathers around the user's face and assists in encasing the user's hair within the protective hair covering 912. When the hair bonnet 910 is used as a bag, the adjustable cords 952 assist in closing the opening of the bag.

The hair bonnet 910 also includes a flap 958 that is configured to extend across the opening of the hair bonnet 910 when the hair bonnet 910 is used as a bag. The flap 958 is further configured to attach to the pocket 948 so that the opening of the hair bonnet 910 as a bag and the opening of the pocket 948 can be opened and closed. For example, the openings of the hair bonnet 910 as a bag and the opening of the pocket 948 are preferably closed when a user is walking around with the hair bonnet 910 to avoid theft or loss of items within the hair bonnet 910. The flap 958 and the pocket 948 include attachment mechanisms 950A, 950B that can reversibly attach to each other. For example, and in some embodiments, the attachment mechanisms 950A, 950B are strips of hook and loop fasteners, snaps, buttons and holes, G-hooks and loops, S-hooks and loops, or any other suitable fasteners.

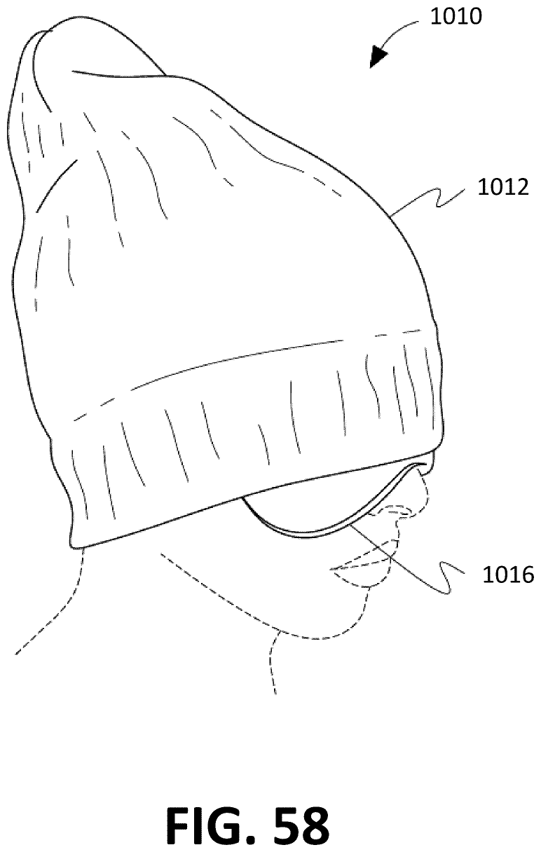

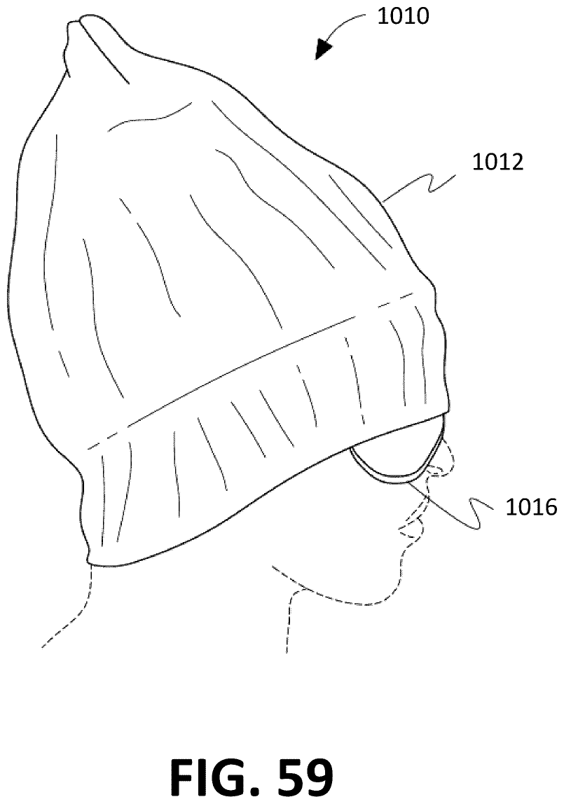

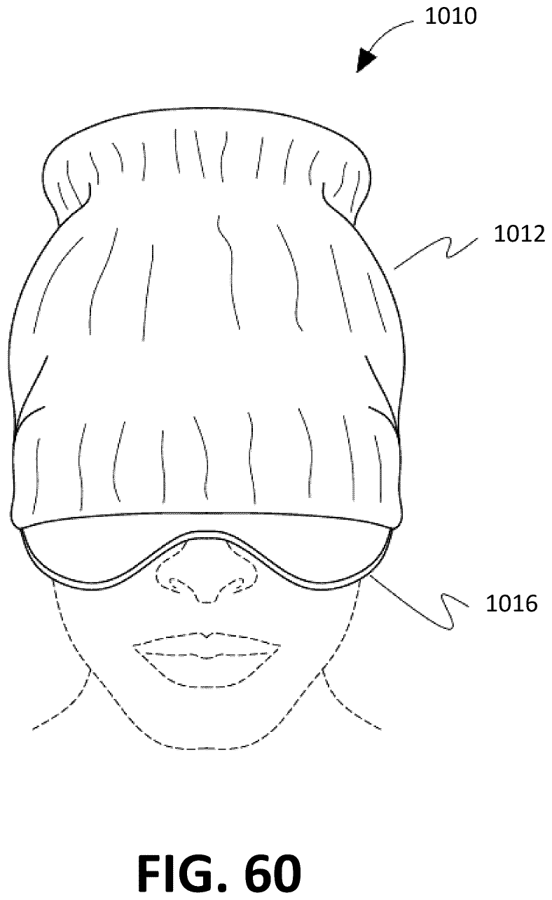

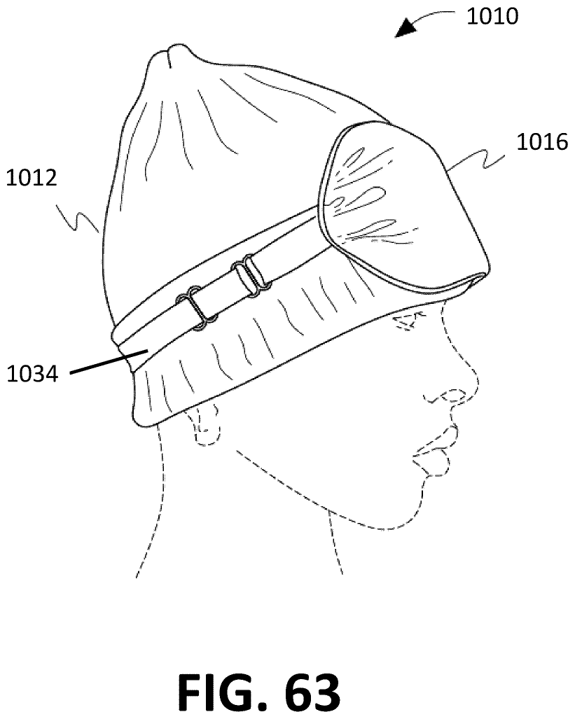

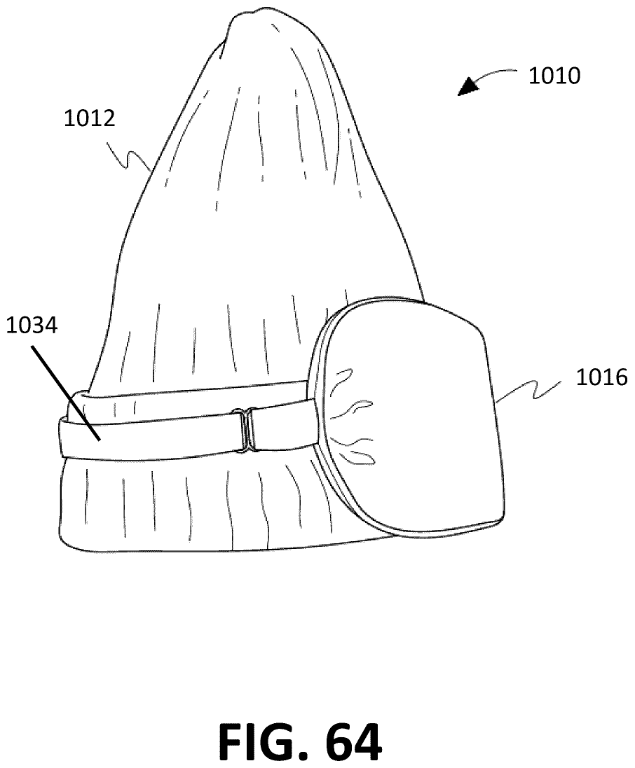

FIGS. 58-64 illustrate an alternative configuration for a hair bonnet 1010 relative to the hair bonnets 10, 110, 210, 310, 410, 410′, 510, 610, 710, 810, 910 of FIGS. 1-57. In FIGS. 58-64, the reference numerals are listed as 1000-series reference numerals that represent structures and features that are similar to the structures and features of the hair bonnets 10, 110, 210, 310, 410, 410′, 510, 610, 710, 810, 910 of FIGS. 1-57 that have two-digit, 100-series, 200-series, 300-series, 400-series, 400′-series, 500-series, 600-series, 700-series, 800-series, or 900-series reference numerals, respectively.

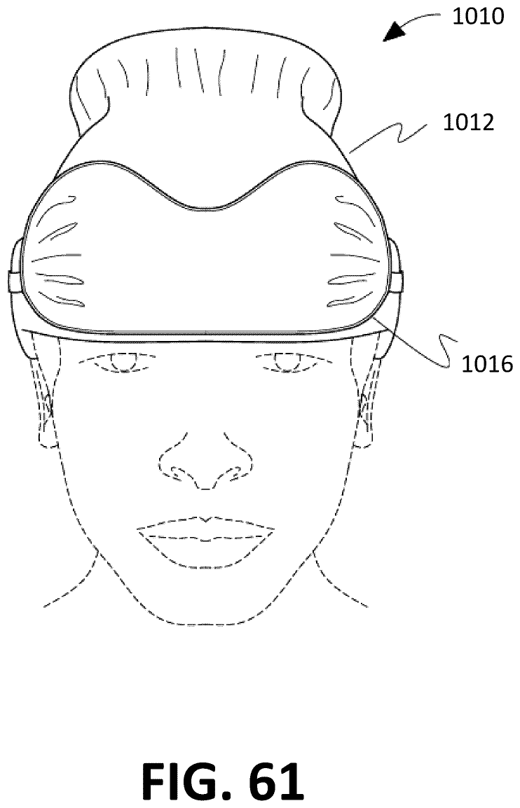

The hair bonnet 1010 includes a protective hair covering 1012 and an eye mask 1016 coupled to the protective hair covering 1012. The protective hair covering 1012 is shaped and configured to be worn as a beanie. As shown in FIGS. 58-60, the eye mask 1016 is kept in place over a user's eyes by the brim of the protective hair covering 1012. As shown in FIGS. 61-64, the brim of the protective hair covering 1012 and the eye mask 1016 may be flipped upwards onto a user's forehead when the user does not desire to wear the eye mask 1016. For example, the user may flip the eye mask 1016 upwards when the user is awake and flip the eye mask 1016 downwards when the user is asleep or is trying to sleep.

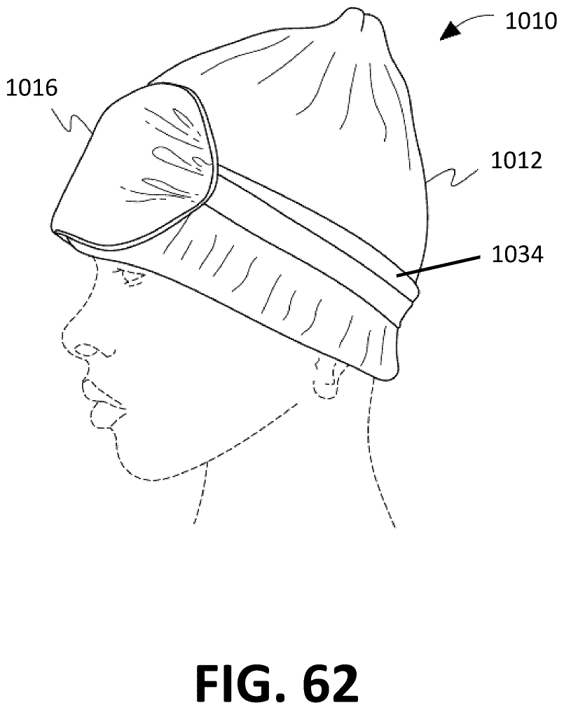

As shown in FIGS. 62-64, the hair bonnet 1010 also includes an adjustable strap 1034 that assists in tightening and/or loosening the eye mask 1016 on the user's head. The adjustable strap 1034 can be adjusted by any suitable means. For example, and in some embodiments, the adjustable strap 1034 includes a flexible material, a ring, a slide, a hook, a tie down strap, a tension strap, and/or a buckle.

Embodiments of the invention provide an innovative wellness eye mask bonnet designed to promote hair health protection, enhance rest, and effect pure relaxation. By providing full hair and eye coverage, employing advanced light-blocking technology, incorporating soft and comfortable materials, offering an adjustable fit and secure closure, and potentially integrating therapeutic elements and acoustic dampening features, embodiments of the invention reduce a user's hair and eye stress.

The invention has been described in relation to particular examples, which are intended in all respects to be illustrative rather than restrictive. Those skilled in the art will appreciate that many different combinations will be suitable for practicing the present invention. Other implementations of the invention will be apparent to those skilled in the art from consideration of the specification and practice of the invention disclosed herein. Various aspects and/or components of the described embodiments may be used singly or in any combination. It is intended that the specification and examples be considered as exemplary only, with a true scope and spirit of the invention being indicated by the following claims.

What is claimed is:

1. A hair bonnet comprising:
a protective hair covering, the protective hair covering comprising fabric; and
an eye mask coupled to the protective hair covering.

2. The hair bonnet of claim 1, wherein the protective hair covering is configured to prevent moisture loss.

3. The hair bonnet of claim 1, wherein the protective hair covering is configured to prevent hair breakage.

4. The hair bonnet of claim 1, wherein the eye mask is integral with the protective hair covering.

5. The hair bonnet of claim 1, wherein the eye mask is removably coupled to the protective hair covering.

6. The hair bonnet of claim 1, wherein the protective hair covering is hydrophobic.

7. The hair bonnet of claim 1, wherein the protective hair covering is shaped and sized to envelop hair of a user.

8. The hair bonnet of claim 1, wherein the eye mask is configured to block at least 50% of light.

9. The hair bonnet of claim 1, wherein the eye mask comprises padding.

10. The hair bonnet of claim 1, wherein the eye mask comprises an opening and further comprises an insert configured to be inserted into the opening.

11. The hair bonnet of claim 10, wherein the insert comprises a gel eye mask.

12. The hair bonnet of claim 10, wherein the insert comprises a cooling pack.

13. The hair bonnet of claim 10, wherein the insert comprises a heating pack.

14. The hair bonnet of claim 1, further comprising a securing mechanism coupled to the protective hair covering.

15. The hair bonnet of claim 14, wherein the securing mechanism comprises a first strap and a second strap.

16. The hair bonnet of claim 1, further comprising an ear mask coupled to the protective hair covering.

17. The hair bonnet of claim 16, wherein the ear mask is integral with the protective hair covering.

18. The hair bonnet of claim 16, wherein the ear mask is removably coupled to the protective hair covering.

19. The hair bonnet of claim 16, wherein the ear mask is configured to block at least 50% of sound.

20. A kit for a hair bonnet comprising:
a hair bonnet comprising:
a protective hair covering, the protective hair covering comprising fabric; and
an eye mask coupled to the protective hair covering, the eye mask comprising an opening; and
an insert configured to be inserted into the opening.
